(19) United States
(12) Patent Application Publication
(10) Pub. No.: US 2003/0124158 A1
(43) Pub. Date: Jul. 3, 2003

(54) Low-emulsifier or emulsifier-free systems of the oil-in-water type with a content of stabilizers and an amino-substituted hydroxybenzophenone

(76) Inventors: Thomas Heidenfelder, Dannstadt (DE); Thomas Wunsch, Speyer (DE); Valerie Andre, Ludwigshafen (DE)

Correspondence Address: Herbert B. Keil, KEIL & WEINKAUF, 1350 Connecticut Ave., N.W., Washington, DC, 20036, US

(21) Appl. No.: 10/232,376

(22) Filed: Sep. 3, 2002

(30) Foreign Application Priority Data
Sep. 7, 2001 (DE) 10143964.4

(51) Int. Cl. A61K 31/24; A61K 7/00

(52) U.S. Cl. 424/401; 514/541

(57) ABSTRACT

Cosmetic or dermatological preparations which represent finely disperse systems of the oil-in-water type, comprising

a) an oil phase,

b) a water phase,

c) one or more stabilizers,

d) at most 2.00% by weight of one or more emulsifiers, and

e) an amino-substituted hydroxybenzophenone of the formula I

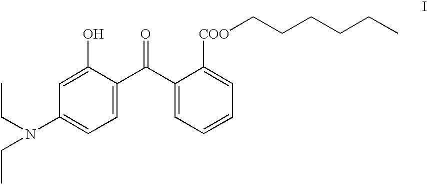

Low-emulsifier or emulsifier-free systems of the oil-in-water type with a content of stabilizers and an amino-substituted hydroxybenzophenone

The present invention relates to emulsifier-free or low-emulsifier finely disperse systems of the oil-in-water type, preferably as cosmetic or dermatological preparations.

Emulsions are generally understood as meaning heterogeneous systems which consist of two liquids which are immiscible or only miscible with one another to a limited extent, and which are usually referred to as phases. In an emulsion, one of the two liquids is dispersed in the other liquid in the form of very fine droplets.

If the two liquids are water and oil and oil droplets are finely dispersed in water, then this is an oil-in-water emulsion (O/W emulsion, e.g. milk). The basic character of a O/W emulsion is defined by the water. In the case of a water-in-oil emulsion (W/O emulsion, e.g. butter), the principle is reversed, and the basic character is here determined by the oil.

In order to achieve permanent dispersion of one liquid in another, it is necessary to add an interface-active substance (emulsifier) in the case of emulsions in the traditional sense. Emulsifiers have an amphiphilic molecular structure, consisting of a polar (hydrophilic) and a nonpolar (lipophilic) molecular moiety, which are spatially separate from one another. In simple emulsions, finely disperse droplets of one phase (water droplets in W/O emulsions or lipid vesicles in O/W emulsions), surrounded by an emulsifier shell, are present in a second phase. Emulsifiers reduce the interfacial tension between the phases by positioning themselves at the interface between two liquids. At the oil/water phase boundary they form interfacial films, which prevent irreversible coalescence of the droplets. Emulsions are frequently stabilized using emulsifier mixtures.

Traditional emulsifiers can be divided into ionic (anionic, kationic and amphoteric) and nonionic, depending on their hydrophilic molecular moiety:

The best known example of an anionic emulsifier is soap, a term usually used to refer to the water-soluble sodium or potassium salts of saturated or unsaturated higher fatty acids.

Important representatives of the cationic emulsifiers are the quaternary ammonium compounds.

The hydrophilic molecular moiety of nonionic emulsifiers often consists of glycerol, polyglycerol, sorbitans, carbohydrates or polyoxyethylene glycols and is usually linked to the lipophilic molecular moiety via ester and ether bonds. The lipophilic molecular moiety usually consists of fatty alcohols, fatty acids or isofatty acids.

By varying the structure and the size of the polar and the nonpolar molecular moiety, the lipophilicity and hydrophilicity of emulsifiers can be varied within wide limits.

A decisive factor for the stability of an emulsion is the correct choice of emulsifiers. In this connection, the characteristics of all substances present in the system are to be taken into account. If, for example, skincare emulsions are considered, then polar oil components and, for example, UV filters lead to instabilities. As well as the emulsifiers, therefore, other stabilizers are generally also used which, for example increase the viscosity of the emulsion and/or act as protective colloid.

Emulsions represent an important product type in the field of cosmetic and/or dermatological preparations.

Cosmetic preparations are essentially used for skin care. Skin care in the cosmetics sense is primarily that the natural function of the skin as a barrier against environmental influences (e.g. soil, chemicals, microorganisms) and against the loss of endogenous substances (e.g. water, natural fats, electrolytes) is strengthened or restored. Impairment of this function may lead to increased resorption of toxic or allergenic substances or to attack by microorganisms and, consequently, to toxic or allergic skin reactions.

Another aim of skin care is to compensate for the loss by the skin of lipids and water caused by daily washing. This is particularly important when the natural regeneration ability is insufficient. Furthermore, skincare products should protect against environmental influences, in particular against sun and wind, and delay skin aging.

Cosmetic preparations are also used as deodorants. Such formulations serve to eliminate body odor which arises when fresh perspiration, which is in itself odorless, is decomposed by microorganisms.

Medicinal topical compositions generally comprise one or more medicaments in an effective concentration. For the sake of simplicity, in order to distinguish unambiguously between cosmetic and medicinal use and corresponding products, reference is made to the legal provisions of the Federal Republic of Germany (e.g. Cosmetics Directive, Foods and Drugs Act).

Per se, the use of customary emulsifiers in cosmetic or dermatological preparations is safe. Nevertheless, emulsifiers, as ultimately any chemical substance, can in individual cases cause allergic reactions or reactions based on hypersensitivity of the user.

Thus, for example, it is known that certain photodermatoses are simultaneous exposure to sunlight. Such photodermatoses are also called “Mallorca acne”. There has thus been no lack of attempts to reduce the amount of customary emulsifiers to a minimum, ideally even completely.

As well as the emulsions described, the prior art recognizes further emulsifier-free, finely disperse cosmetic or dermatological preparations, which are generally referred to as hydrodispersions. Hydrodispersions represent dispersions of a liquid, semisolid or solid internal (discontinuous) lipid phase in an external aqueous (continuous) phase.

In the case of hydrodispersions of a liquid lipid phase in an external aqueous phase, the stability can, for example, be ensured by constructing, in the aqueous phase, a gel structure in which the lipid droplets are stably suspended. German Laid-Open Specification 44 25 268 describes stable, finely disperse, emulsifier-free cosmetic or dermatological preparations of the oil-in-water type which, in addition to an oil and a water phase, comprise one or more thickeners from the group of acrylic acid polymers, polysaccharides and alkyl ethers thereof, although for these thickeners it must not be possible to measure a reduction in the interfacial tension.

Based on similar hydrodispersions, German Laid-Open Specification 43 03 983 discloses cosmetic or dermatological light protection formulations which are essentially free from emulsifiers, inorganic micropigments which serve as UV filter substances being incorporated into the lipid phase of the hydrodispersion.

Although on the one hand there is a large number of possibilities for designing entirely emulsifier-free preparations, on the other hand a certain low emulsifier content is sometimes not entirely undesired since the absence of emsulfiers presents technical difficulties during the incorporation of many raw materials.

It is an object of the present invention to enrich the prior art with cosmetic or dermatological preparations by means of which, with toleration of a low content of emulsifiers, stable preparations with a good feel on the skin are obtained.

We have found that this object is achieved by cosmetic or dermatological preparations which represent finely disperse systems of the oil-in-water type, comprising

a) an oil phase,

b) a water phase,

c) one or more stabilizers,

d) at most 2.00% by weight of one or more emulsifiers and

e) an amino-substituted hydroxybenzophenone of the formula I

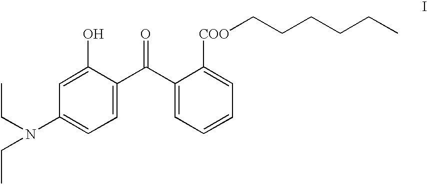

and optionally further cosmetic or pharmaceutical auxiliaries, additives and/or active ingredients. According to the invention, it is particularly advantageous if the preparations are emulsifier-free, but at most comprise 0.1 to 2.00% by weight, preferably 0.5 to 1.5% by weight, based on the total weight of the preparations, of one or more emulsifiers.

The stabilizers used in the preparations according to the invention are preferably representatives from the class of substance of hydrocolloids.

“Hydrocolloid” is the technological abbreviation for the more correct name “hydrophilic colloid”. Hydrocolloids are macromolecules which have a largely linear structure and have intermolecular forces of interaction which permit secondary and primary valence bonds between the individual molecules and thus the formation of a recticular structure. Some are water-soluble natural or synthetic polymers which, in aqueous systems, form gels or viscous solutions. They increase the viscosity of the water by either binding water molecules (hydration) or else by absorbing and encapsulating the water into their interwoven macromolecules, at the same time as restricting the mobility of the water. Such water-soluble polymers represent a large group of chemically very different natural and synthetic polymers whose common feature is their solubility in water or aqueous media. A prerequisite for this is that these polymers have a number of hydrophilic groups sufficient for solubility in water and are not too greatly crosslinked.

The hydrophilic groups can be nonionic, anionic or cationic in nature, for example as follows:

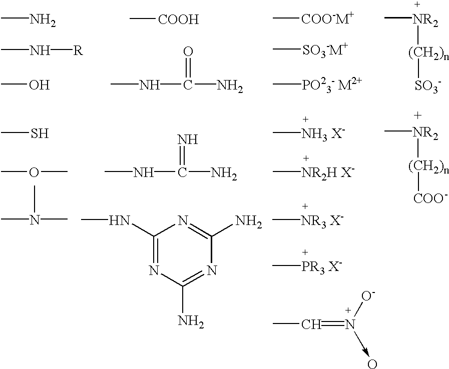

The group of the cosmetically and dermatologically relevant hydrocolloids can be divided as follows into:

organic, natural compounds, such as, for example, agar agar, carrageen, tragacanth, gum arabic, alginates, pectins, polyoses, guar flour, carob bean flour, starch, dextrins, gelatins, casein;

organic, modified natural substances, such as, for example, carboxymethylcellulose and other cellulose ethers, hydroxyethylcellulose and hydroxypropylcellulose and microcrystalline cellulose;

organic, completely synthetic compounds, such as, for example, polyacrylic and polymethacrylic compounds, vinyl polymers, polycarboxylic acids, polyethers, polyimines, polyamides, polyurethanes;

inorganic compounds, such as, for example, polysilicic acids, clay minerals, such as montmorillonites, zeolites, silicas.

Advantageous stabilizers are ethylcelluloses. Ethylcelluloses are characterized by the following structure:

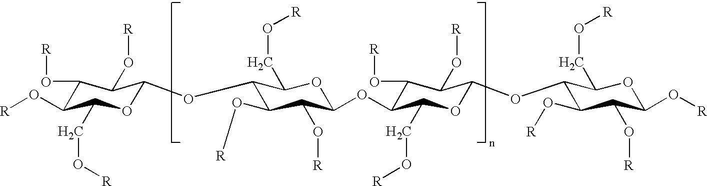

Here, R can either be ethyl groups or hydrogen atoms.

The degree of ethylation in the ethylcellulose is advantageously 2.0 to 3.0, corresponding to 40 to 55%, preferably 48.0 to 49.5% ethylation. The average molecular mass is preferably to be chosen such that the viscosity of a 5% strength solution in a mixture of 80 parts of toluene and 20 parts of ethanol at 25° C. is 3 to 110 mPas, preferably 9 to 11 mPas. The average molar mass is particularly advantageously 100,000 to 400,000 g/mol.

The content of ethylcellulose in the preparations according to the invention is preferably 0.1 to 10% by weight, based on the total weight of the preparations. Such products are available, for example, under the trade name ETHOCEL® Standard 10 Premium (Dow Chemicals).

Microcrystalline cellulose is an advantageous hydrocolloid for the purposes of the present invention. It is available, for example, from “FMC Corporation Food and Pharmaceutical Products” under the trade name Avicel®. A particularly advantageous product for the purposes of the present invention is the grade Avicel® RC-591, which is a modified microcrystalline cellulose which is made up of 89% microcrystalline cellulose and 11% sodium carboxymethylcellulose. Other commercial products from this class of raw material are Avicel® RC/CL, Avicel® CE-15, Avicel® 500.

Further hydrocolloids used advantageously are, for example, methylcelluloses, which is the name for the methylethers of cellulose. They are characterized by the following structural formula

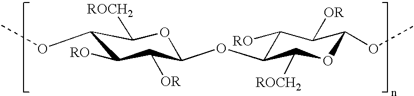

in which R can be a hydrogen or a methyl group. Particularly advantageous for the purposes of the present invention are the cellulose mixed ethers, which are generally likewise referred to as methylcelluloses, which contain, in addition to a predominating content of methyl groups, also 2-hydroxyethyl, 2-hydroxypropyl or 2-hydroxybutyl groups. Particular preference is given to (hydroxypropyl)methyl-celluloses, for example those available under the trade name Methocel® E4M from Dow Chemical Comp.

Also advantageous according to the invention is sodium carboxymethylcellulose, the sodium salt of the glycolic ether of cellulose, for which R in the above structural formula may be hydrogen and/or CH₂—COONa. Particular preference is given to sodium carboxymethylcellulose available under the trade name Natrosol® Plus 330 CS from Aqualon and also referred to as cellulose gum.

Also preferred for the purposes of the present invention is xanthan (CAS No. 11138-66-2), also called xanthan gum, which is an anionic heteropolysaccharide which is generally formed by fermentation from maize sugar and is isolated as potassium salt. It is produced by xanthomonas campestris and some other species under aerobic conditions and has a molecular weight of from 2×10⁶ to 24×10⁶. Xanthan is formed from a chain having β-1,4-bonded glucose (cellulose) with side chains. The structure of the subgroups consists of glucose, mannose, glucuronic acid, acetate and pyruvate. The number of pyruvate units determines the viscosity of the xanthan.

A further advantageous gel former for the purposes of the present invention is also carrageen, a gel-forming extract with a similar structure to agar, of North Atlantic red algae which belong to the Florideae (Chondrus crispus and Gigartina stellata).

The term carrageen is frequently used for the dried algae product and carrageenan for the extract thereof. The carrageen precipitated from the hot-water extract of the algae is a colorless to sand-colored powder with a molecular weight range from 100,000 to 800,000 and a sulfate content of about 25%. Carrageen, which is very readily soluble in warm water, forms a thixotropic gel upon cooling, even if the water content is 95-98%. The rigidity of the gel is effected by the double helix structure of the carrageen.

In the case of carrageenan, three principle constituents are differentiated: The gel-forming κ fraction consists of D-galactose 4-sulfate and 3,6-anhydro-α-D-galactose, which has alternate glycoside bonds in the 1,3- and 1,4 position (by contrast, agar contains 3,6-anhydro-α-L-galactose). The nongelling λ fraction is composed of 1,3-glycosidically linked D-galactose 2-sulfate and 1,4-bonded D-galactose-2,6-disulfate radicals, and is readily soluble in cold water. ι-Carrageenan, composed of D-galactose 4-sulfate in 1,3 bond and 3,6-anhydro-α-D-galactose 2-sulfate in 1,4 bond, is both water-soluble and also gel-forming. The nature of cations which are present (K⁺, NH₄⁺, Na⁺, Mg²⁺, Ca²⁺) also influences the solubility of the carrageens.

Polyacrylates are gelling agents likewise to be used preferably for the purposes of the present invention. Polyacrylates advantageous according to the invention are acrylate-alkyl acrylate copolymers, in particular those chosen from the group of carbomers or carbopols (Carbopol® is actually a registered trademark of B. F. Goodrich Company). In particular, the acrylate-alkyl acrylate copolymers advantageous according to the invention are characterized by the following structure:

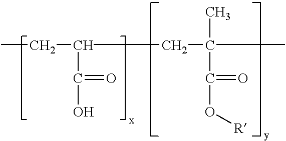

where R′ is a long-chain alkyl radical, and x and y represent numbers which symbolize the respective stoichiometric proportion of each of the comonomers.

According to the invention, particular preference is given to acrylate copolymers and/or acrylate-alkyl acrylate copolymers which are available under the trade names Carbopol® 1382, Carbopol® 981 and Carbopol® 5984 from B. F. Goodrich Company and very particular preference is give to polyacrylates from the group of Carbopol grades 980, 981, 1382, 2984, 5984 and Carbomer 2001.

Also advantageous are copolymers of C₁₀-₃₀-alkyl acrylates and one or more monomers of acrylic acid, of methacrylic acid or esters thereof which are crosslinked with an allyl ether of sucrose or an allyl ether of pentaerythritol.

Compounds which carry the INCI name “Acrylates/C₁₀-₃₀ Alkyl Acrylate Crosspolymer” are likewise advantageous. Particularly advantageous are those polymers available under the trade names Pemulen® TR1 and Pemulen® TR2 from B. F. Goodrich Company.

Compounds which carry the INCI name ammonium acryloyldimethyltaurates/vinylpyrrolidone copolymers are advantageous.

According to the invention, the ammonium acryloyldimethyl taurate/vinylpyrrolidone copolymers have the empirical formula [C₇H₁₆N₂SO₄]ₙ, [C₆H₉NO]ₘ, which corresponds to the following statistical structure

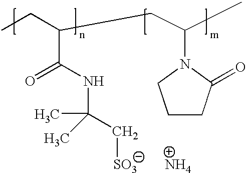

Preferred species for the purposes of the present invention are listed in the Chemical Abstracts under the Registry numbers 58374-69-9, 13162-05-5 and 88-12-0 and are available under the trade name Aristoflex® AVC from Clariant GmbH.

Also advantageous are copolymers/crosspolymers comprising Acryloyldimethyl Taurate, such as, for example, Simugel® EG or Simugel® EG from Seppic S.A.

Further completely synthetic hydrocolloids to be used according to the invention are:

A. anionic polyurethanes which are soluble or dispersible in water and which are advantageously obtainable from

Aa) at least one compound which contains two or more active hydrogen atoms per molecule,

Ab) at least one diol containing acid or salt groups and

Ac) at least one diisocyanate.

The component Aa) is, in particular, a diol, aminoalcohol, diamine, polyesterol, polyetherol with a number-average molecular-weight of in each case up to 3000, or mixtures thereof, where up to 3 mol % of said compounds may be replaced by triols or triamines. Preference is given to diols and polyesterdiols. In particular, the component Aa) comprises at least 50% by weight, based on the total weight of the component Aa), of a polyesterdiol. Suitable polyesterdiols are all those which are customarily used for the preparation of polyurethanes, in particular the reaction products of phthalic acid and diethylene glycol, isophthalic acid and 1,4-butanediol, isophthalic acid/adipic acid and 1,6-hexanediol, and adipic acid and ethylene glycol or 5-NaSO₃-isophthalic acid, phthalic acid, adipic acid and 1,6-hexanediol.

Examples of diols which can be used are ethylene glycol, propylene glycol, butylene glycol, neopentyl glycol, polyetherols, such as polyethylene glycols having molecular weights up to 3000, block copolymers of ethylene oxide and propylene oxide with number-average molecular weights of up to 3000 or block copolymers of ethylene oxide, propylene oxide and butylene oxide which contain the copolymerized alkylene oxide units in randomly distributed manner or in the form of blocks. Preference is given to ethylene glycol, neopentyl glycol, di-, tri-, tetra-, penta- or hexaethylene glycol. Other diols which can be used are poly(u-hydroxycarboxylic acid)diols.

Suitable aminoalcohols are, for example, 2-aminoethanol, 2-(N-methylamino)ethanol, 3-aminopropanol or 4-aminobutanol.

Exmaples of suitable diamines are ethylenediamine, propylenediamine, 1,4-diaminobutane and 1,6-diaminohexane, and α,ω-diamines which can be prepared by amination of polyalkylene oxides with ammonia.

Component Ab) is, in particular, dimethylolpropanoic acid or compounds of the formulae

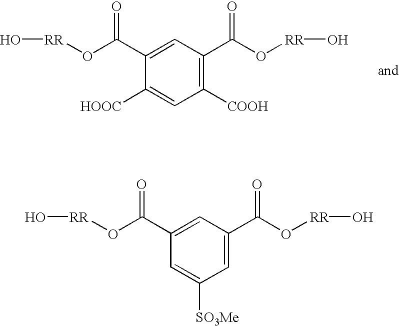

where RR is in each case a C₂-C₁₈-alkylene group and Me is Na or K.

Component Ac) is, in particular, hexamethylene diisocyanate, isophorone diisocyanate, methyldiphenyl isocyanate (MDI) and/or tolylene diisocyanate.

The polyurethanes are obtainable by reacting the compounds of groups Aa) and Ab) under an inert-gas atmosphere in an inert solvent at temperatures of from 70 to 130° C. with the compounds of group Ac). This reaction can be carried out, where appropriate, in the presence of chain extenders in order to prepare polyurethanes with relatively high molecular weights. As is customary in the preparation of polyurethanes, the components [(Aa)+(Ab)]:Ac are advantageously used in the molar ratio of from 0.8 to 1.1:1. The acid number of the polyurethanes is determined by the composition and the concentration of the compounds of component (Ab) in the mixture of components (Aa) and (Ab).

The polyurethanes have K values according to H. Fikentscher (determined in 0.1% strength by weight solutions in N-methylpyrrolidone at 25° C. and pH 7) of from 15 to 100, preferably 25 to 50.

The K value, also referred to as the intrinsic viscosity, is a parameter which is easy to determine by means of viscosity measurements of polymer solutions and is therefore frequently used in the industrial sector for characterizing polymers.

The polyurethanes containing acid groups are, after neutralization (partial or complete), water-soluble or dispersible without the aid of emulsifiers. The salts of the polyurethanes generally have better solubility or dispersibility in water than the unneutralized polyurethanes. Bases which can be used for the neutralization of the polyurethanes are alkali metal bases, such as sodium hydroxide solution, potassium hydroxide solution, soda, sodium hydrogencarbonate, potassium carbonate or potassium hydrogen carbonate and alkaline earth metal bases, such as calcium hydroxide, calcium oxide, magnesium hydroxide or magnesium carbonate, and ammonia and amines. 2-Amino-2-methylpropanol, diethylaminopropylamine and triisoproanolamine have proven particularly useful for the neutralization of the polyurethanes containing acid groups. The neutralization of the polyurethanes containing acid groups can also be carried out using mixtures of two or more bases, e.g. mixtures of sodium hydroxide solution and triisopropanolamine. Depending on the intended use, neutralization may be partial, e.g. 20 to 40%, or complete, i.e. 100%.

These polymers and their preparation are described in more detail in DE-A-42 25 045, to the entire scope of which reference is hereby made.

B. Water-soluble or -dispersible cationic polyurethanes and polyureas of

Ba) at least one diisocyanate, which may have already been reacted beforehand with one or more compounds which contain two or more active hydrogen atoms per molecule, and

Bb) at least one diol, primary or secondary amino alcohol, primary or secondary diamine or primary or secondary triamine with one or more tertiary, quaternary or protonated tertiary amino nitrogen atoms.

Preferred diisocyanates are as given above under A). Compounds with two or more active hydrogen atoms are diols, aminoalcohols, diamines, polyesterols, polyamidediamines and polyetherols. Suitable compounds of this type are as given above under A).

The polyurethanes are prepared as described above under A). Charged cationic groups can be produced in the polyureas from the tertiary amino nitrogen atoms present either by protonation, e.g. with carboxylic acids, such as lactic acid, or by quaternization, e.g. with alkylating agents, such as C₁ to C₄-alkyl halides or sulfates. Examples of such alkylating agents are ethyl chloride, ethyl bromide, methyl chloride, methyl bromide, dimethyl sulfate and diethyl sulfate.

These polymers and their preparation are described in more detail in DE-A-42 41 118, to the entire scope of which reference is hereby made.

C. Linear polyurethanes with carboxylate groups of

Ca) a 2,2-hydroxymethyl-substituted carboxylic acid of the formula

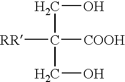

in which RR′ is a hydrogen atom or a C₁-C₂₀-alkyl group, which is used in an amount which suffices for 0.35 to 2.25 milliequivalents of carboxyl groups to be present in the polyurethane per g of polyurethane,

Cb) 10 to 90% by weight, based on the weight of the polyurethane, of one or more organic compounds with not more than two active hydrogen atoms and

Cc) one or more organic diisocyanates.

The carboxyl groups present in the polyurethane are, finally, at least partially neutralized with a suitable base. These polymers and their preparation are described in EP-A-619 111, to the entire scope of which reference is hereby made.

D. Carboxyl-containing polycondensation products of anhydrides of tri- or tetracarboxylic acids and diols, diamines or aminoalcohols (polyesters, polyamides or polyester amides).

These polymers and their preparation are described in more detail in DE-A-42 24 761, to the entire scope of which reference is hereby made.

E. Polyacrylates and polymethacrylates, as are described in more detail in DE-A-43 14 305, 36 27 970 and 29 17 504. Reference is hereby made to these publications in their entirety.

The polymers used according to the invention preferably have a K value of from 15 to 100, preferably 25 to 50. The polymers are generally present in the composition according to the invention in an amount in the range from 0.2 to 20% by weight, based on the total weight of the compositions. The salt is used in an amount effective for improving the exchangeability of the polymers. The salt is generally used in an amount of from 0.02 to 10% by weight, preferably 0.05 to 5% by weight and in particular 0.1 to 3% by weight, based on the total weight of the composition.

As inorganic thickeners, it is possible, for example, to choose representatives from the group of modified or unmodified, naturally occurring or synthetic phyllosilicates.

Although it is entirely favorable to use pure components, it may, however, be advantageous for mixtures of different modified and/or unmodified phyllosilicates to be present in the compositions according to the invention.

For the purposes of this application, phyllosilicates are understood as meaning silicates and alumosilicates in which the silicate or aluminate units are linked together via three Si—O— or Al—O— bonds and form a wavy sheet or layer structure. The fourth Si—O— or Al—O— valence is saturated by cations. Relatively weak electrostatic interactions, e.g. hydrogen bridge bonds, exist between the individual layers. The layer structure, meanwhile, is largely defined by strong, covalent bonds.

The stochiometry of the sheet silicates is (Si₂O₅²⁻) for pure silicate structures and (AlₘSi²⁻ₘO₅^{(2+m)-}) for alumosilicates.

m is a number greater than zero and less than 2.

If pure silicates are not present, but alumosilicates, the circumstance that each Si⁴⁺ group replaced by Al³⁺ requires another singly charged cation to neutralize the charge is to be taken into account.

The charge balance is preferably evened out by H⁺, alkali metal ions or alkaline earth metal ions. Aluminum as counterion is also known and advantageous. In contrast to the alumosilicates, these compounds are called aluminum silicates. “Aluminum alumosilicates”, in which aluminum is present both in the silicate network, and also as counterion, are known and sometimes advantageous for the present invention.

Phyllosilicates are well documented in the literature, e.g. in the “Lehrbuch der Anorganischen Chemie” [Textbook of Inorganic Chemistry], A. F. Hollemann, E. Wiberg and N. Wiberg, 91st-100th Ed., Walter de Gruyter—Verlag 1985, passim, and “Lehrbuch der Anorganischen Chemie”, H.Remy, 12^{th} Ed., Akademische Verlagsgesellschaft, Leipzig 1965, passim. The layer structure of montmorillonite is given in Römpps Chemie-Lexikon, Franckh'sche Verlagshandlung W. Keller & Co., Stuttgart, 8^{th} Ed., 1985, p. 2668 f.

| | |
|---|---|
| Montmorillonite | Na_{0.33}((Al_{1.67}Mg_{0.33})(OH)₂(Sᵢ₄O₁₀)) |
| often simplified: | Al₂O₃*4SiO₂*H₂O*nH₂O or |
| | Al₂[(OH)₂/Si₄O₁₀].n H₂O |
| Kaolinite | Al₂(OH)₄(Si₂O₅) |
| Illite | (K,H₃O)_{y}(Mg₃(OH)₂(Si_{4-y}Al_{y}O₁₀)) or |
| | (K,H₃O)_{y}(Al₂(OH)₂(Si_{4-y}Al_{y}O₁₀)) |
| | where y = 0.7-0.9 |
| Beidellite | (Ca,Na)_{0.3}(Al₂(OH)₂(Al_{0.5}Si_{3.5}O₁₀)) |
| Nontronite | Na_{0.33}(Fe₂(OH)₂(Al_{0.33}Si_{3.67}O₁₀)) |
| Saponite | (Ca,Na)_{0.33}((Mg,Fe)₃(OH)₂(Al_{0.33}Si_{3.67}O₁₀)) |
| Hectorite | Na_{0.33}((Mg,Li)₃(OH,F)₂(Si₄O₁₀)) |

Montmorillonite is the main mineral of the naturally occurring bentonites.

Very advantageous inorganic gel formers for the purposes of the present invention are aluminum silicates, such as the montmorillonites (bentonites, hectorites and derivatives thereof, such as quaternium-18 bentonite, quaternium-18 hectorites, stearalkonium bentonites and stearalkonium hectorites), and also magnesium-aluminum silicates (Veegum® grades), and sodium-magnesium silicates (Laponite® grades).

Montmorillonites represent clay minerals which are a type of dioctahedral smectites, and are masses which swell in water, but do not become plastic. The layer packets in the three-layer structure of the montmorillonites can swell as a result of reversible incorporation of water (in a 2- to 7-fold amount) and other substances, such as, for example, alcohols, glycols, pyridine, α-picoline, ammonium compounds, hydroxy-aluminosilicate ions etc.

The chemical formula given above is only approximate: since montmorillonites have a large capacity for ion exchange, Al can be replaced by Mg, Fe²⁺, Fe³⁺, Zn, Pb (e.g. from harmful substances in waste waters), Cr, and also Cu and others. The resulting negative charge of the octahedral layers is compensated by cations, in particular Na⁺ (sodium montmorillonite) and Ca²⁺ (calcium montmorillonite is only swellable to a very small extent) in interlayer positions.

Synthetic magnesium silicates and/or bentonites advantageous for the purposes of the present invention are sold, for example, by Süd-Chemie under the trade name Optigel®.

An advantageous aluminum silicate advantageous for the purposes of the present invention is sold, for example, by R. T. Vanderbilt Comp., Inc., under the trade name Veegum®. The various Veegum® grades, which are all advantageous according to the invention, are characterized by the following compositions

| | (regular | | | | |
|---|---|---|---|---|---|
| | grade) | HV | K | HS | S-728 |
| SiO₂ | 55.5 | 56.9 | 64.7 | 69.0 | 65.3 |
| MgO | 13.0 | 13.0 | 5.4 | 2.9 | 3.3 |
| Al₂O₃ | 8.9 | 10.3 | 14.8 | 14.7 | 17.0 |
| Fe₂O₃ | 1.0 | 0.8 | 1.5 | 1.8 | 0.7 |
| CaO | 2.0 | 2.0 | 1.1 | 1.3 | 1.3 |
| Na₂O | 2.1 | 2.8 | 2.2 | 2.2 | 3.8 |
| K₂O | 1.3 | 1.3 | 1.9 | 0.4 | 0.2 |
| Ashing loss | 11.1 | 12.6 | 7.6 | 5.5 | 7.5 |

These products swell in water to form viscous gels, which have an alkaline reaction. The organophilization of montmorillonite or bentonites (exchange of the interlayer cations for quaternary alkylammonium ions) produces products (bentones) which are preferably used for dispersion in organic solvents and oils, fats, ointments, inks, surface coatings and in detergents.

Bentone® is a trade name for various neutral and chemically inert gelling agents which are constructed from long-chain, organic ammonium salts and specific types of montmorillonite. Bentones swell in organic media and cause the latter to swell. The gels are resistant in diluted acids and alkalis, although they partially lose their gelling properties upon prolonged contact with strong acids and alkalis. Because of their organophilic character, the bentones are only wettable by water with difficulty.

The following Bentone® grades are sold, for example, by Kronos Titan: Bentone® 27, an organically modified montmorillonite, Bentone® 34 (dimethyldioctylammonium bentonite), which is prepared in accordance with U.S. Pat. No. 2,531,427 and, because of its lipophilic groups, swells more readily in lipophilic medium than in water, Bentone® 38, an organically modified montmorillonite, a cream-colored to white powder, Bentone® LT, a purified clay mineral, Bentone® Gel MIO, an organically modified montmorillonite which is supplied as a very fine suspension in mineral oil (SUS-71) (10% bentonite, 86.7% mineral oil and 3.3% wetting agent), Bentone® Gel IPM, an organically modified bentonite which is suspended in isopropyl myristate (10% bentonite, 86.7% isopropylmyristate, 3.3% wetting agent), Bentone® Gel CAO, an organically modified montmorillonite which is taken up in castor oil (10% bentonite, 86.7% castor oil and 3.3% wetting agent), Bentone® Gel Lantrol, an organically modified montmorillonite which, in paste form, is intended for the further processing, in particular for the preparation, of cosmetic compositions; 10% bentonite, 64.9 Lantrol (wool wax oil), 22.0 isopropyl myristate, 3.0 wetting agent and 0.1 propyl p-hydroxybenzoate, Bentone® Gel Lan I, a 10% strength Bentone® 27 paste in a mixture of wool wax USP and isopropyl palmitate, Bentone® Gel Lan II, a bentonite paste in pure liquid wool wax, Bentone® Gel NV, a 15% strength Bentone® 27 paste in dibutyl phthalate, Bentone® Gel OMS, a bentonite paste in Shellsol T., Bentone® Gel OMS 25, a bentonite paste in isoparaffinic hydrocarbons (Idopar® H), Bentone® Gel IPP, a bentonite paste in isopropyl palmitate.

The total amount of one or more hydrocolloids preferably used as stabilizers in the finished cosmetic or dermatological preparations is advantageously chosen to be less than 5% by weight, preferably between 0.05 and 3.0% by weight, particularly preferably between 0.1 and 1.0% by weight, based on the total weight of the preparations.

The preparations according to the invention comprise an amino-substituted hydroxybenzophenone of the formula I as component e).

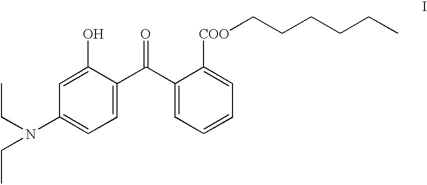

This class of substance is described, inter alia, in DE-A-199 17 906. Compound I is characterized in particular by its good solubility in oil, its good processability into cosmetic preparations and by a good feel on the skin.

The amount of compound of the formula I used in the finished cosmetic or dermatological preparations is advantageously chosen from the range 0.01% by weight to 20% by weight, preferably from 0.1 to 10% by weight, particularly preferably from 1 to 7% by weight, based on the total weight of the preparations.

Basic constituents of the preparations according to the invention which may be used are:

water or aqueous solutions

aqeuous ethanolic solutions

natural oils and/or chemically modified natural oils and/or synthetic oils;

fats, waxes and other natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids;

alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products.

In particular, mixtures of the abovementioned solvents are used.

For the purposes of the present disclosure, the expression “lipids” is sometimes used as a generic term for fats, oils, waxes and the like, said expression being entirely commonplace to the person skilled in the art. The terms “oil phase” and “lipid phase” are also used synonymously.

Oils and fats differ from one another in their polarity, which is difficult to define. It has already been proposed to adopt the interfacial tension toward water as a measure of the polarity index of an oil or of an oily phase. Then, the lower the interfacial tension between this oily phase and water, the greater the polarity of the oily phase in question. According to the invention, the interfacial tension is regarded as one possible measure of the polarity of a given oil component.

The interfacial tension is the force which acts on an imaginary line one meter in length in the interface between two phases. The physical unit for this interfacial tension is conventionally calculated from the force/length relationship and is usually expressed in mN/m (millinewtons divided by meters). It has a positive sign if it endeavours to reduce the interface. In the converse case, it has a negative sign. For the purposes of the present invention, lipids are regarded as polar if their interfacial tension toward water is less than 30 mN/m.

Polar oils are for example those from the group of lecithins and of fatty acid triglycerides, namely the triglycerol esters of saturated and/or unsaturated, branched and/or unbranched alkanecarboxylic acids having a chain length of from 8 to 24, in particular 12 to 18, carbon atoms. The fatty acid triglycerides can, for example, advantageously be chosen from the group of synthetic, semisynthetic and natural oils, such as, for example, olive oil, sunflower oil, soya oil, groundnut oil, rapeseed oil, almond oil, palm oil, coconut oil, castor oil, wheatgerm oil, grapeseed oil, thistle oil, evening primrose oil, macadamia nut oil and the like, provided the condition required in the main claim is observed.

Other polar oil components can be chosen from the group of esters of saturated and/or unsaturated, branched and/or unbranched alkanecarboxylic acids having a chain length of from 3 to 30 carbon atoms and saturated and/or unsaturated, branched and/or unbranched alcohols having a chain length of from 3 to 30 carbon atoms, and from the group of esters of aromatic carboxylic acids and saturated and/or unsaturated, branched and/or unbranched alcohols having a chain length of from 3 to 30 carbon atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semisynthetic and natural mixtures of such esters, such as, for example, jojoba oil.

In addition, the oily phase can be advantageously chosen from the group of dialkyl ethers, the group of saturated or unsaturated, branched or unbranched alcohols. It is particularly advantageous if the oily phase of the preparations according to the invention contains C₁₂-₁₅-alkyl benzoate or consists entirely of the latter.

In addition, the oil phase can advantageously be chosen from the group of Guerbet alcohols. Guerbet alcohols are named after Marcel Guerbet who described their preparation for the first time. They are formed according to the equation

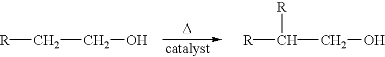

by oxidation of an alcohol to an aldehyde, by aldol condensation of the aldehyde, elimination of water from the aldol and hydrogenation of the allyl aldehyde. Guerbet alcohols are liquid even at low temperatures and effect virtually no skin irritations. They can be used advantageously as fatting, superfatting and also refatting constituents in skincare and haircare compositions.

The use of Guerbet alcohols in cosmetics is known per se. Such species are then in most cases characterized by the structure

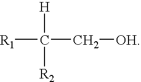

Here, R₁ and R₂ are usually unbranched alkyl radicals.

According to the invention, the Guerbet alcohol(s) is/are advantageously chosen from the group in which

R₁ is propyl, butyl, pentyl, hexyl, heptyl or octyl and

R₂ is hexyl, heptyl, octyl, nonyl, decyl, undecyl, dodecyl, tridecyl or tetradecyl.

Guerbet alcohols preferred according to the invention are 2-butyloctanol with the following chemical structure

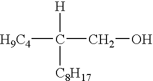

which is available, for example, under the trade name Isofol® 12 from Condea Chemie GmbH, and 2-hexyldecanol with the following chemical structure

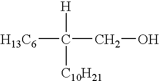

which is available, for example, under the trade name Isofol® 16 from Condea Chemie GmbH.

Mixtures of Guerbet alcohols according to the invention can also be used advantageously according to the invention. Mixtures of 2-butyloctanol and 2-hexyldecanol are available, for example, under the trade name Isofol® 14 from Condea Chemie GmbH.

The total amount of Guerbet alcohols in the finished cosmetic or dermatological preparations is advantageously chosen from the range up to 25.0% by weight, preferably 0.5 to 15.0% by weight, based on the total weight of the preparations.

Any mixtures of such oil and wax components can also be used advantageously for the purposes of the present invention. It may also be advantageous to use waxes, for example cetyl palmitate, as the sole lipid component of the oil phase.

Nonpolar oils are, for example, those which are chosen from the group of branched and unbranched hydrocarbons and hydrocarbon waxes, in particular Vaseline (petrolatum), paraffin oil, squalane and squalene, polyolefins and hydrogenated polyisobutenes. Of the polyolefins, polydecenes are the preferred substances.

Fatty and/or wax components which are to be used advantageously according to the invention can be chosen from the group of vegetable waxes, animal waxes, mineral waxes and petrochemical waxes. Examples which are favorable according to the invention are candelilla wax, carnauba wax, japan wax, esparto grass wax, cork wax, guaruma wax, rice germ oil wax, sugar cane wax, berry wax, ouricury wax, montan wax, jojoba wax, shea butter, beeswax, shellac wax, spermaceti, lanolin (wool wax), uropygial grease, ceresin, ozokerite (earth wax), paraffin waxes and microcrystalline waxes provided the conditions required in the main claim are observed.

Other advantageous fatty and/or wax components are chemically modified waxes and synthetic waxes, such as, for example, those obtainable under the trade names Syncrowax® HRC (glyceryl tribehenate) and Syncrowax® AW 1C (C₁₈-C₃₆ fatty acid) from CRODA GmbH, and montan ester waxes, Sasol waxes, hydrogenated jojoba waxes, synthetic or modified beeswaxes (e.g. dimethicone copolyol beeswax and/or C₃₀-₅₀ alkyl beeswax), polyalkylene waxes, polyethylene glycol waxes, but also chemically modified fats, such as, for example, hydrogenated vegetable oils (for example hydrogenated castor oil and/or hydrogenated coconut fatty glycerides), triglycerides, such as, for example, trihydroxystearin, fatty acids, fatty acid esters, and glycol esters, such as, for example, C₂₀-C₄₀-alkyl stearate, C₂₀-C₄₀-alkylhydroxystearoyl stearate and/or glycol montanate. Also advantageous are certain organosilicon compounds, which have similar physical properties to the specified fatty and/or wax components, such as, for example, stearoxytrimethylsilane provided the conditions required in the main claim are observed.

According to the invention, the fatty and/or wax components can be present either individually or as a mixture.

Any desired mixtures of such oil and wax components can also be used advantageously for the purposes of the present invention.

The oily phase is advantageously chosen from the group consisting of 2-ethylhexyl isostearate, octyldodecanol, isotridecyl isononanoate, isoeicosane, 2-ethylhexyl cocoate, C₁₂-C₁₅-alkyl benzoate, caprylic/capric triglyceride, dicaprylyl ether provided the conditions required in the main claim are observed.

Particularly advantageous mixtures are those of octyldodecanol, caprylic/capric triglyceride, dicaprylyl ether or mixtures of C₁₂-C₁₅-alkyl benzoate and 2-ethylhexyl isostearate, those of C₁₂-C₁₅-alkyl benzoate and isotridecyl isononanoate, and those of C₁₂-C₁₅-alkyl benzoate, 2-ethylhexyl isostearate and isotridecyl isononanoate, provided the conditions required in the main claim are observed.

Table 1 below lists lipids which are advantageous according to the invention as individual substances and also as mixtures with one another. The corresponding interfacial tensions toward water are given in the last column. It is, however, also advantageous to use mixtures of greater or lesser polar components and the like.

| Trade name | INCI name | (m/Nm) |
|---|---|---|
| Isofol ® 14 T | Butyl Decanol + Hexyl Decanol + Hexyl | 27.6 |
| | Octanol + Butyl Octanol | |
| Isofol ® 16 | Hexyl Decanol | 24.3 |
| Eutanol ® G | Octyldodecanol | 24.8 |
| Cetiol ® OE | Dicaprylyl Ether | 22.1 |
| Miglyol ® 812 | Caprylic/Capric Triglyceride | 21.3 |
| Cegesoft ® C24 | Octyl Palmitate | 23.1 |
| Isopropyl | Isopropyl Stearate | 21.9 |
| stearate | | |
| Estol ® 1540 EHC | Octyl Octanoate | 30.0 |
| Finsolv ® TN | C₁₂ -C₁₅ Alkyl Benzoate | 21.8 |
| Cetiol ® SN | Cetearyl Isonoanoate | 28.6 |
| Dermofeel ® BGC | Butylene Glycol Dicaprylate/Dicapate | 21.5 |
| Trivent ® OCG | Tricaprylin | 20.2 |
| MOD | Octyldodeceyl Myristate | 22.1 |
| Cosmacol ® ETI | Di-C₁₂-C₁₃ Alkyl Tartrate | 29.4 |
| Miglyol ® 829 | Caprylic/Capric Diglyceryl Succinate | 29.5 |
| Prisorine ® 2036 | Octyl Isostearate | 29.7 |
| Tegosoft ® SH | Stearyl Heptanoate | 28.7 |
| Abil ® Wax 9840 | Cetyl Dimethicone | 25.1 |
| Cetiol ® LC | Coco-Caprylate/Caprate | 24.8 |
| IPP | Isopropyl Palmitate | 22.5 |
| Luvitol ® EHO | Cetearyl Octanoate | 28.6 |
| Cetiol ® 868 | Octyl Stearate | 28.4 |

It may likewise be advantageous to choose some or all of the oil phase of the preparations according to the invention from the group of cyclic and/or linear silicones which are also referred to for the purposes of the present disclosure as “silicone oils”. Such silicones or silicone oils may be present as monomers which are generally characterized by structural elements as follows:

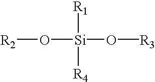

Silicones having two or more siloxyl units which are to be used advantageously according to the invention are generally characterized by structural elements as follows:

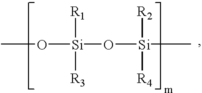

where the silicon atoms may be substituted by identical or different alkyl radicals and/or aryl radicals, which are represented in general terms by the radicals R₁ to R₄, where the number of different radicals is not necessarily limited to 4. m may assume values from 2 to 200,000.

Cyclic silicones to be used advantageously according to the invention are generally characterized by the structural elements as follows

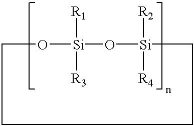

where the silicon atoms may be substituted by identical or different alkyl radicals and/or aryl radicals, which are represented here in general terms by the radicals R₁ to R₄, where the number of different radicals is not necessarily limited to 4. can assume values of 3/2 to 20. Fractional values for n take into consideration that uneven numbers of siloxyl groups may be present in the cycle.

Phenyltrimethicone is advantageously chosen as silicone oil. Other silicone oils, for example dimethicone, phenyldimethicone, cyclomethicone (octamethylcyclotetrasiloxane), for example hexamethylcyclotrisiloxane, polydimethylsiloxane, poly(methylphenylsiloxane), cetyldimethicone, behenoxydimethicone can also be used advantageously for the purposes of the present invention.

Mixtures of cyclomethicone and isotridecyl isononanoate, and those of cyclomethicone and 2-ethylhexyl isostearate are also advantageous.

It is, however, also advantageous to choose silicone oils of similar constitution, such as the compounds referred to above whose organic side chains have been derivatized, for example polyethoxylated and/or polypropoxylated. These include, for example, polysiloxane-polyalkyl-polyether copolymers such as cetyldimethicone copolyol, (cetyldimethicone copolyol (and) polyglyceryl-4 isostearate (and) hexyl laurate).

If, then, a small amount of emulsifiers is to be used, W/O emulsifiers and/or O/W emulsifiers can be used, for example advantageously chosen from the group of the following substances which generally act as W/O emulsifiers:

lecithin, lanolin, microcrystalline wax (Cera microcristallina) in a mixture with paraffin oil (Paraffinum liquidum), ozokerite, hydrogenated castor oil, polyglyceryl-3 oleate, wool wax acid mixtures, wool wax alcohol mixtures, pentaerythrithyl isostearate, polyglyceryl-3 diisostearate, beeswax (Cera alba) and stearic acid, sodium dihydroxycetylphosphate in a mixture with isopropyl hydroxycetyl ether, methylglucose dioleate, methylglucose dioleate in a mixture with hydroxystearate and beeswax, mineral oil in a mixture with petrolatum and ozokerite and glyceryl oleate and lanolin alcohol, petrolatum in a mixture with ozokerite and hydrogenated castor oil and glyceryl isostearate and polyglyceyl-3 oleate, PEG-7 hydrogenated castor oil, ozokerite and hydrogenated castor oil, polyglyceryl-4 isostearate, polyglyceryl-4 isostearate in a mixture with cetyldimethicone copolyol and hexyl laurate, laurylmethicone copolyol, cetyldimethicone copolyol, acrylate/C₁₀-C₃₀-alkyl acrylate crosspolymer, Poloxamer 101, polyglyceryl-2 dipolyhydroxystearate, polyglyceryl-3 diisostearate, polyglyceryl-4 dipolyhydroxystearate, PEG-30 dipolyhydroxystearate, diisostearoyl polyglyceryl-3 diisostearate, polyglyceryl-2 dipolyhydroxystearate, polyglyceryl-3 dipolyhydroxystearate, polyglyceryl-4 dipolyhydroxystearate, polyglyceryl-3 dioleate.

If desired, one or more emulsifiers can be used, chosen from the group of the following substances which generally act as O/W emulsifiers:

Glyceryl stearate in a mixture with ceteareth-20, ceteareth-25, ceteareth-6 in a mixture with stearyl alcohol, cetylstearyl alcohol in a mixture with PEG-40 castor oil and sodium cetylstearyl sulfate, triceteareth-4 phosphate, sodium cetylstearyl sulfate, lecithin trilaureth-4 phosphate, laureth-4 phosphate, stearic acid, propylene glycol stearate SE, PEG-25 hydrogenated castor oil, PEG-54 hydrogenated castor oil, PEG-6 caprylic/capric glycerides, glyceryl oleate in a mixture with propylene glycol, ceteth-2, ceteth-20, polysorbate 60, glyceryl stearate in a mixture with PEG-100 stearate, laureth-4, ceteareth-3, isostearyl glyceryl ether, cetylstearyl alcohol in a mixture with sodium cetylstearyl sulfate, laureth-23, steareth-2, glyceryl stearate in a mixture with PEG-30 stearate, PEG-40 stearate, glycol distearate, PEG-22 dodecyl glycol copolymer, polyglyceryl-2 PEG-4 stearate, ceteareth-20, methylglucose sesquistearate, steareth-10, PEG-20 stearate, steareth-2 in a mixture with PEG-8 distearate, steareth-21, steareth-20, isosteareth-20, PEG-45/dodecyl glycol copolymer, methoxy-PEG-22/dodecyl glycol copolymer, PEG-20 glyceryl stearate, PEG-8 beeswax, polyglyceryl-2 laurate, isostearyl diglyceryl succinate, stearamidopropyl PG dimonium chloride phosphate, glyceryl stearate SE, ceteth-20, triethyl citrate, PEG-20 methylglucose sesquistearate, ceteareth-12, glyceryl stearate citrate, cetyl phosphate, triceteareth-4 phosphate, trilaureth-4 phosphate, polyglyceryl methylglucose distearate, potassium cetyl phosphate, isosteareth-10, polyglyceryl-2 sesquiisostearate, ceteth-10, oleth-20, isoceteth-20, glyceryl stearate in a mixture with ceteareth-20, ceteareth-12, cetylstearyl alcohol and cetyl palmitate, cetylstearyl alcohol in a mixture with PEG-20 stearate, PEG-30 stearate, PEG-40 stearate, PEG-100 stearate.

It may also be advantageous for the purposes of the present invention, particularly when the oil phase of the preparations consists at least partially of silicone oils, to use silicone emulsifiers. The silicone emulsifiers may advantageously be chosen from the group of interface-active substances from the group of alkylmethicone copolyols and/or alkyl dimethicone copolyols, particularly from the group of compounds characterized by the following chemical structure:

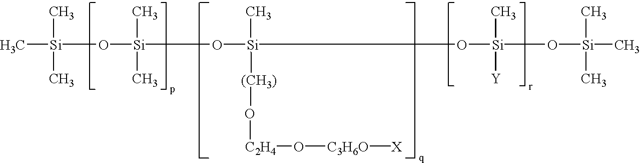

in which X and Y, independently of one another, are chosen from the group H and the branched and unbranched alkyl groups, acyl groups and alkoxy groups having 1 to 24 carbon atoms, p is a number from 0 to 200, q is a number from 1 to 40, and r is a number from 1 to 100.

An example of silicone emulsifiers which are to be used particularly advantageously for the purposes of the present invention are dimethicone copolyols, which are sold by Th. Goldschmidt AG under the trade names ABIL® B 8842, ABIL® B 8843, ABIL® B 8847, ABIL® B 8851, ABIL® B 8852, ABIL® B 8863, ABIL® B 8873 and ABIL® B 88183.

Another example of interface-active substances to be used particularly advantageously for the purposes of the present invention is cetyldimethicone copolyol, which is sold by Th. Goldschmidt AG under the trade name ABIL® EM 90.

Another example of interface-active substances to be used particularly advantageously for the purposes of the present invention is the cyclomethiconedimethicone copolyol, which is sold by Th. Goldschmidt AG under the trade name ABIL® EM 97.

In addition, an emulsifier which has proven especially advantageous is laurylmethicone copolyol, which is available under the trade name Dow Corning® 5200 Formulation Aid from Dow Corning Ltd.

If, then, a small amount of silicone emulsifiers is to be used, their total amount in the cosmetic or dermatological preparations according to the invention is advantageously in the range from 0.1 to 2.0% by weight, preferably 0.5 to 1.5% by weight, based on the total weight of the preparations.

Preparations according to the invention for the purposes of the present invention, e.g. in the form of a skin protection cream, a skin lotion, a cosmetic milk, for example in the form of a sunscreen cream or a sun protection milk, are advantageous and comprise, for example, fats, oils, waxes and/or other fatty substances, and water and, if desired, one or more emulsifiers as are customarily used for such a type of formulation.

Just as preparations of liquid and solid consistency are used as cosmetic cleansing lotions or cleansing creams, the preparations according to the invention may also represent sprayable cleansing preparations (“cleansing sprays”), which are used, for example, for removing make-up or as mild washing lotion—optionally also for blemished skin. Such cleansing preparations can advantageously further be used as “rinse-off preparations”, which are rinsed off from the skin following application.

It is naturally known to the person skilled in the art that demanding cosmetic compositions are in most cases inconceivable without customary auxiliaries and additives. These include, for example, bodying agents, fillers, perfume, dyes, emulsifiers, additional active ingredients such as vitamins and proteins, light protection agents, stabilizers, insect repellents, alcohol, water, salts, antimicrobially, proteolytically or keratolytically active substances etc.

Corresponding requirements apply mutatis mutandis to the formulation of medicinal preparations.

Accordingly, cosmetic or topical dermatological preparations for the purposes of the present invention can, depending on the composition, be used, for example, as skin protection cream, cleansing milk, sun screen lotion, nourishing cream, day cream or night cream etc. It is in some instances possible and advantageous to use the compositions according to the invention as bases for pharmaceutical formulations.

It is likewise advantageous to use the preparations according to the invention for decorative cosmetics (make-up formulations).

Also favorable are those cosmetic and dermatological preparations which are in the form of a sun screen. In addition to the active ingredient used according to the invention, these preferably additionally comprise at least one broadband filter and/or at least one UVA filter substance and/or at least one UVB filter substance and/or at least one inorganic pigment.

It is, however, also advantageous for the purposes of the present invention to create cosmetic and dermatological preparations whose main purpose is not protection against sunlight, but which nevertheless have a content of UV protection substances. Thus, for example, UV-A and/or UV-B filter substances are usually incorporated into day creams.

Advantageous broadband filters, UV-A or UV-B filter substances are, for example, bis-resorcinyltriazine derivatives of the following structure:

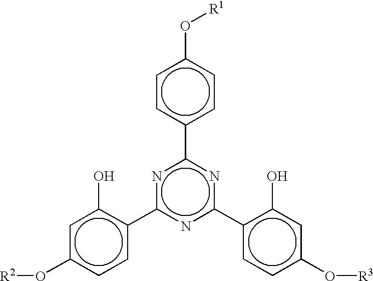

where R¹, R² and R³, independently of one another, are chosen from the group of branched and unbranched alkyl groups having 1 to 10 carbon atoms or represent a single hydrogen atom. Particular preference is given to

2,4-bis-{[4-(2-ethylhexyloxy)-2-hydroxy]phenyl}-6-(4-methoxy-phenyl)-1,3,5-triazine (INCI: Aniso Triazine), which is available under the trade name Tinosorb® S from CIBA-Chemikalien GmbH.

Other UV filter substances which have the structural formula

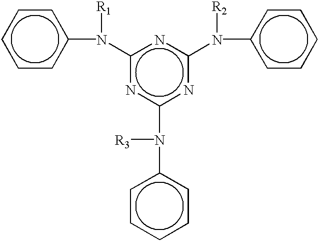

are advantageous UV filter substances for the purposes of the present invention, for example the s-triazine derivatives described in European Laid-Open Specification EP 570 838 A1, the chemical structure of which is given by the generic formula

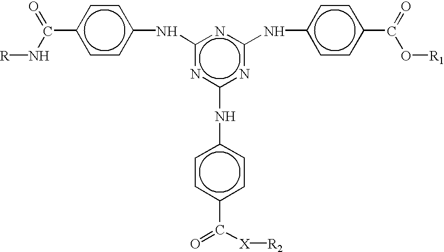

where

R is a branched or unbranched C₁-C₁₈-alkyl radical, a C₅-C₁₂-cycloalkyl radical, optionally substituted by one or more C₁-C₄-alkyl groups,

X is an oxygen atom or an NH group,

R₁ is a branched or unbranched C₁-C₁₈-alkyl radical, a C₅-C₁₂-cycloalkyl radical, optionally substituted by one or more C₁-C₄-alkyl groups, or a hydrogen atom, an alkali metal atom, an ammonium group or a group of the formula

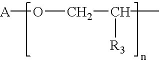

in which

A is a branched or unbranched C₁-C₁₈-alkyl radical, a C₅-C₁₂-cycloalkyl or aryl radical, optionally substituted by one or more C₁-C₄-alkyl groups,

R₃ is a hydrogen atom or a methyl group,

n is a number from 1 to 10,

R₂ is a branched or unbranched C₁-C₁₈-alkyl radical, a C₅-C₁₂-cycloalkyl radical, optionally substituted by one or more C₁-C₄-alkyl groups, if X is the NH group, and a branched or unbranched C₁-C₁₈-alkyl radical, a C₅-C₁₂-cycloalkyl radical, optionally substituted by one or more C₁-C₄-alkyl groups, or a hydrogen atom, an alkali metal atom, an ammonium group or a group of the formula

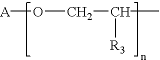

in which

A is a branched or unbranched C₁-C₁₈-alkyl radical, a C₅-C₁₂-cycloalkyl or aryl radical, optionally substituted by one or more C₁-C₄-alkyl groups,

R₃ is a hydrogen atom or a methyl group,

n is a number from 1 to 10,

if X is an oxygen atom.

A particularly preferred UV filter substance for the purposes of the present invention is also an asymmetrically substituted s-triazine whose chemical structure is given by the formula

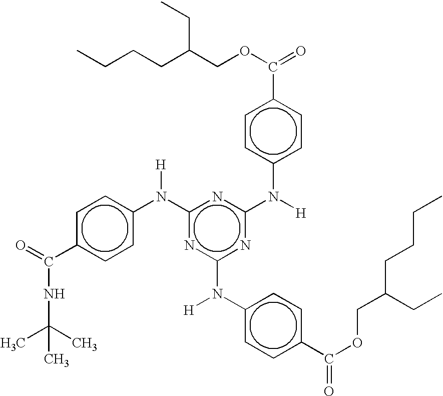

which is also referred to below as dioctylbutylamidotriazone (INCI: Diethylhexylbutamidotriazone) and is available under the trade name UVASORB® HEB from Sigma 3V.

Also advantageous for the purposes of the present invention is a symmetrically substituted s-triazine, tris(2-ethylhexyl) 4,4′,4″-(1,3,5-triazine-2,4,6-triyltriimino)trisbenzoate, synonym:

2,4,6-tris-[anilino(p-carbo-2′-ethyl-1′-hexyloxy)]-1,3,5-triazine (INCI: ethylhexyl triazone), which is sold by BASF Aktiengesellschaft under the trade name UVINUL® T 150.

European Laid-Open Specification 775 698 also describes preferred bis-resorcinyltriazine derivatives, the chemical structure of which is given by the generic formula

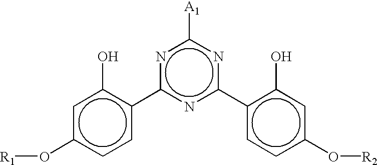

where R₁ and R₂ represent, inter alia, C₃-C₁₈-alkyl or C₂-C₁₈-alkenyl and Al is an aromatic radical.

Also advantageous for the purposes of the present invention are

2,4-bis{[4-(3-sulfonato)-2-hydroxypropyloxy)-2-hydroxy]phenyl}-6-(4-methoxyphenyl)-1,3,5-triazine sodium salt,

2,4-bis{[4-(3-(2-propyloxy)-2-hydroxypropyloxy)-2-hydroxy]phenyl}-6-(4-methoxyphenyl)-1,3,5-triazine,

2,4-bis{[4-(2-ethylhexyloxy)-2-hydroxy]phenyl}-6-[4-(2-methoxy-ethylcarboxyl)phenylamino]-1,3,5-triazine,

2,4-bis{[4-(3-(2-propyloxy)-2-hydroxypropyloxy)-2-hydroxy]phenyl}-6-[4-(2-ethylcarboxyl)phenylamino]-1,3,5-triazine,

2,4-bis{[4-(2-ethylhexyloxy)-2-hydroxy]phenyl}-6-(1-methylpyrrol-2-yl)-1,3,5-triazine,

2,4-bis{([4-tris(trimethyl-siloxysilylpropyloxy)-2-hydroxy]phenyl}-6-(4-methoxyphenyl)-1,3,5-triazine,

2,4-bis{[4-(2″-methylpropenyloxy)-2-hydroxy]phenyl}-6-(4-methoxy-phenyl)-1,3,5-triazine and

2,4-bis{[4-(1′,1′,1′,3′,5′,5′,5′-heptamethylsiloxy-2″-methyl-propyloxy)-2-hydroxy]phenyl}-6-(4-methoxyphenyl)-1,3,5-triazine.

The total amount of one or more triazine derivatives optionally used in the finished cosmetic or dermatological preparations is advantageously chosen from the range 0.01% by weight to 15% by weight, preferably from 0.1 to 10% by weight, in each case based on the total weight of the preparations.

Advantageous sulfonated, water-soluble UV filters for the purposes of the present invention are:

Phenylene-1,4-bis(2-benzimidazyl)-3,3′-5,5′-tetrasulfonic acid, which is characterized by the following structure:

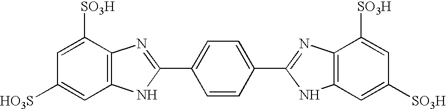

and its salts, particularly the corresponding sodium, potassium or triethanolammonium salts, in particular the phenylene-1,4-bis(2-benzimidazyl)-3,3′-5,5′-tetrasulfonic acid bis-sodium salt

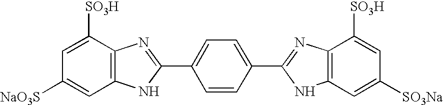

with the INCI name bisimidazylate (CAS No.: 180898-37-7), which is available, for example, under the trade name Neo Heliopan® AP from Haarmann & Reimer.

A further advantageous sulfonated UV filter for the purposes of the present invention are the salts of

2-phenylbenzimidazol-5-sulfonic acid, and its sodium, potassium or its triethanolammonium salt, and the sulfonic acid itself

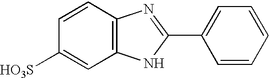

with the INCI name Phenylbenzimidazole Sulfonic Acid (CAS No. 27503-81-7), which is available, for example, under the trade name Eusolex® 232 from Merck or under Neo Heliopan® Hydro from Haarmann & Reimer.

A further advantageous sulfonated UV filter is 3,3′-(1,4-phenylenedimethylene)bis(7,7-dimethyl-2-oxo-bicyclo-[2.2.1]hept-1-ylmethanesulfonic acid, and its sodium, potassium or its triethanolammonium salt, and the sulfonic acid itself:

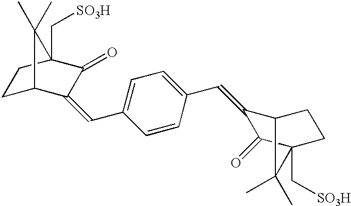

with the INCI name Terephthalidene Dicamphor Sulfonic Acid (CAS No.: 90457-82-2), which is available, for example, under the trade name Mexoryl® SX from Chimex.

Further advantageous water-soluble UV-B and/or broadband filter substances are, for example:

Sulfonic acid derivatives of 3-benzylidenecamphors, such as, for example, 4-(2-oxo-3-bornylidenemethyl)benzenesulfonic acid, 2-methyl-5-(2-oxo-3-bornylidenemethyl)sulfonic acid and salts thereof.

The total amount of one or more sulfonated UV filter substances optionally used in the finished cosmetic or dermatological preparations is advantageously chosen from the range 0.01% by weight to 20% by weight, preferably from 0.1 to 10% by weight, in each case based on the total weight of the preparations.

The UV-B and/or broadband filters may be oil-soluble or water-soluble. Advantageous oil-soluble U-V-B and/or broadband filter substances are, for example:

3-benzylidenecamphor derivatives, preferably 3-(4-methylbenzylidene)camphor, 3-benzylidenecamphor;

4-aminobenzoic acid derivatives, preferably 2-ethylhexyl 4-(dimethylamino)benzoate, amyl 4-(dimethylamino)benzoate, polyethoxyethyl 4-bis (polyethoxy)amino benzoate (available under the trade name Uvinul® P25 from BASF);

Derivatives of benzophenone, preferably

2-hydroxy-4-methoxybenzophenone (available under the trade name Uvinul® M40 from BASF), 2-hydroxy-4-methoxy-4′-methylbenzophenone, 2,2′-dihydroxy-4-methoxybenzophenone, 2-hydroxy-4-methoxybenzophenone-5-sulfonic acid (available under the trade name Uvinul® MS40 from BASF);

and UV filters bonded to polymers.

Particularly advantageous UV filter substances which are liquid at room temperature for the purposes of the present invention are homomenthyl salicylate, 2-ethylhexyl 2-cyano-3,3-diphenylacrylate, 2-ethylhexyl-2-hydroxybenzoate and esters of cinnamic acid, preferably 2-ethylhexyl 4-methoxycinnamate and isopentyl 4-methoxycinnamate.

Homomenthyl salicylate (INCI: Homosalate) is characterized by the following structure:

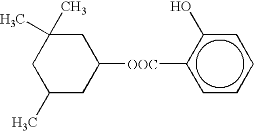

2-ethylhexyl 2-cyano-3,3-diphenylacrylate (INCI: Octocrylene) is available from BASF under the name Uvinul® N 539T and is characterized by the following structure:

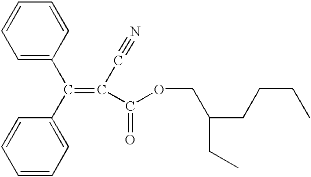

2-ethylhexyl 2-hydroxybenzoate (2-ethylhexyl salicylate, octyl salicylate, INCI: ethylhexyl salicylate) is available, for example, from Haarmann & Reimer under the trade name Neo Heliopan® OS and is characterized by the following structure:

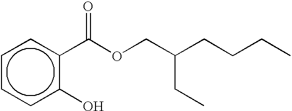

2-ethylhexyl 4-methoxycinnamate (INCI: Ethylhexyl Methoxycinnamate) is available, for example, from BASF under the trade name Uvinul® MC 80 and is characterized by the following structure:

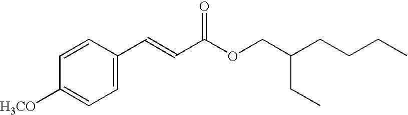

Isopentyl 4-methoxycinnamate (INCI: Isoamyl p-Methoxycinnamate) is available, for example, from Haarmann & Reimer under the trade name Neo Heliopan® E 1000 and is characterized by the following structure:

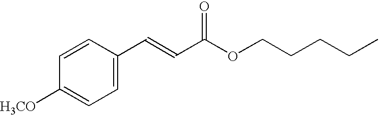

Another advantageous UV filter substance which is liquid at room temperature for the purposes of the present invention is (3-(4-(2,2-bisethoxycarbonylvinyl)phenoxy)propenyl)methyl-siloxane/dimethylsiloxane copolymer, which is available, for example, from Hoffmann-La Roche under the trade name Parsol® SLX.

The total amount of one or more UV filter substances liquid at room temperature optionally used in the finished cosmetic or dermatological preparations is advantageously chosen from the range 0.1% by weight to 30% by weight, preferably from 0.5 to 20% by weight, in each case based on the total weight of the preparations.

Advantageous dibenzoylmethane derivatives for the purposes of the present invention are, in particular, 4-(tert-butyl)-4′-methoxydibenzoylmethane (CAS No. 70356-09-1), which is sold by BASF under the name Uvinul® BMBM and by Merck under the trade name Eusolex® 9020 and is characterized by the following structure:

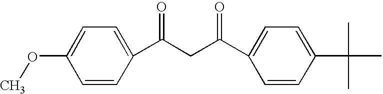

A further advantageous dibenzoylmethane derivative is 4-isopropyldibenzoylmethane (CAS No. 63250-25-9), which is sold by Merck under the name Eusolex® 8020. Eusolex 8020 is characterized by the following structure:

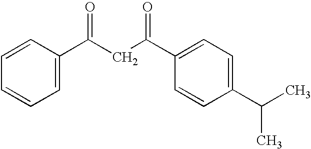

benzotriazoles are characterized by the following structural formula:

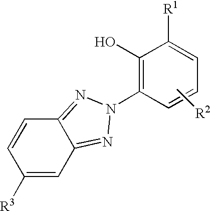

in which

R¹ and R², independently of one another, are linear or branched, saturated or unsaturated, substituted (e.g. substituted by a phenyl radical) or unsubstituted alkyl radicals having 1 to 18 carbon atoms and/or polymer radicals which themselves do not absorb UV rays (such as, for example, silicone radicals, acrylate radicals and the like), and R₃ is chosen from the group H or alkyl radical having 1 to 18 carbon atoms.

An advantageous benzotriazole for the purposes of the present invention is

2,2′-methylenebis(6-(2H-benzotriazol-2-yl)-4-(1,1,3,3-tetramethyl butyl)phenol), a broadband filter which is characterized by the chemical structural formula

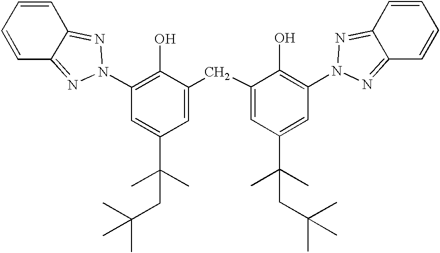

and is available under the trade name Tinosorb® M from CIBA-Chemikalien GmbH.

An advantageous benzotriazole for the purposes of the present invention is also

2-(2H-benzotriazol-2-yl)-4-methyl-6-[2-methyl-3-[1,3,3,3-tetra-methyl-1-[(trimethylsilyl)oxy]disiloxanyl]propyl]phenol (CAS No.: 155633-54-8) with the INCI name Drometrizole Trisiloxane, which is sold by Chimex under the name Mexoryl® XL and is characterized by the following chemical structural formula

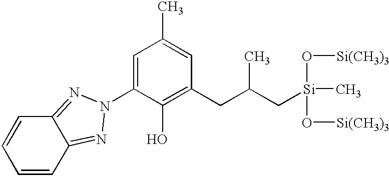

Further advantageous benzotriazoles for the purposes of the present invention are

[2,4′-dihydroxy-3-(2H-benzotriazol-2-yl)-5-(1,1,3,3-tetramethyl-butyl)-2′-n-octoxy-5′-benzoyl]diphenylmethane,

2,2′-methylenebis[6-(2H-benzotriazol-2-yl)-4-(methyl)phenol],

2,2′-methylenebis[6-(2H-benzotriazol-2-yl)-4-(1,1,3,3-tetra-methylbutyl)phenol], 2-(2′-hydroxy-5′-octylphenyl)benzotriazole,

2-(2′-hydroxy-3′,5′-di-t-amylphenyl)benzotriazole and

2-(2′-hydroxy-5′-methylphenyl)benzotriazole.

The total amount of one or more benzotriazoles optionally used in the finished cosmetic or dermatological preparations is advantageously chosen from the range 0.1 to 20% by weight, preferably from 0.5 to 15% by weight, very particularly preferably from 0.5 to 10% by weight, in each case based on the total weight of the preparations.

Another UV-A filter advantageous for the purposes of the present invention is the 2-(4-ethoxyanilinomethylene)propanedicarboxylic diethyl ester, described in EP-A-0 895 776, of the following formula.

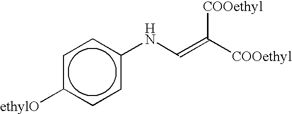

Cosmetic and dermatological preparations according to the invention also advantageously, but not obligatorily, comprise inorganic pigments based on metal oxides and/or other metal compounds which are insoluble or virtually insoluble in water, in particular the oxides of titanium (TiO₂), zinc (ZnO), iron (e.g. Fe2O3), zirconium (ZrO₂), silicon (SiO₂), manganese (e.g. MnO), aluminum (Al₂O₃), cerium (e.g. Ce₂O₃), mixed oxides of the corresponding metals and mixtures of such oxides. These pigments are X-ray amorphous or non-X-ray amorphous. The pigments are particularly preferably based on TiO2.

X-ray amorphous oxide pigments are metal oxides or semimetal oxides which reveal no or no recognizable crystal structure in X-ray diffraction experiments. Such pigments are often obtainable by flame reaction, for example by reacting a metal or semimetal halide with a hydrogen and air (or pure oxygen) in a flame.

In cosmetic, dermatological or pharmaceutical formulations, X-ray amorphous oxide pigments are used as thickeners and thixotropic agents, flow auxiliaries, for emulsion and dispersion stabilization and as carrier substance (for example for increasing volume of finely divided powders).

X-ray amorphous oxide pigments which are known and are often used in cosmetic or dermatological technology are the silicon oxides of the Aerosil® type (CAS No. 7631-86-9). Aerosils®, obtainable from DEGUSSA, are characterized by low particle size (e.g. between 5 and 40 nm), where the particles are to be regarded as spherical particles of very uniform dimension. Macroscopically, Aerosile® are recognizable as loose, white powders. Within the meaning of the present invention, X-ray amorphous silicon dioxide pigments are particularly advantageous and, of these, precisely those of the Aerosil® grade are preferred.

Advantageous Aerosil® grades are, for example, Aerosil® OX50, Aerosil® 130, Aerosil® 150, Aerosil® 200, Aerosil® 300, Aerosil® 380, Aerosil® MOX 80, Aerosil® MOX 170, Aerosil® COK 84, Aerosil® R 202, Aerosil® R 805, Aerosil® R 812, Aerosil® R 972, Aerosil® R 974, Aerosil® R976.

The total amount of one or more X-ray amorphous oxide pigments optionally used in the finished cosmetic or dermatological preparations is advantageously chosen from the range 0.1 to 20% by weight, preferably from 0.5 to 10% by weight, very particularly preferably from 1 to 5% by weight, in each case based on the total weight of the preparations.

According to the invention, the non-X-ray-amorphous inorganic pigments are advantageously in hydrophobic form, i.e. they have been surface-treated to repel water. This surface treatment may involve providing the pigments with a thin hydrophobic layer by methods known per se.

Such a process consists, for example, in producing the hydrophobic surface layer according to a reaction as in

nTiO₂+m(RO)3Si—R′→nTiO₂ (surf.).

Here, n and m are stoichiometric parameters to be used as desired, and R and R′ are the desired organic radicals. Hydrophobicized pigments prepared as in DE-A 33 14 742, for example, are advantageous.

Organic surface coatings for the purposes of the present invention may consist of vegetable or animal aluminum stearate, vegetable or animal stearic acid, lauric acid, dimethylpolysiloxane (also: dimethicone), methylpolysiloxane (methicone), simethicone (a mixture of dimethylpolysiloxane with an average chain length of from 200 to 350 dimethylsiloxane units and silica gel), octyltrimethoxysilane or alginic acid. These organic surface coatings may be present alone, in combination and/or in combination with inorganic coating materials.

Zinc oxide particles suitable according to the invention and predispersions of zinc oxide particles are available under the following trade names from the companies listed:

| Trade name | Coating | Manufacturer |
|---|---|---|
| Z-Cote ® HP1 | 2% Dimethicone | BASF |
| Z-Cote ® | — | BASF |
| ZnO NDM | 5% Dimethicone | H&R |
| MZ-505 S | 5% Methicone | Tayca Corp. |

Suitable titanium dioxide particles and predispersions of titanium dioxide particles are available under the following trade names from the companies listed:

| Trade name | Coating | Manufacturer |
|---|---|---|
| MT-100TV | Aluminum | Tayca Corporation |
| | hydroxide/stearic acid | |
| MT-100Z | Aluminum | Tayca Corporation |
| | hydroxide/stearic acid | |
| Eusolex ® T-2000 | Alumina/simethicone | Merck KgaA |
| Titanium dioxide | Octyltrimethoxysilane | Degussa, BASF |
| T805 (Uvinul ® | | |
| TiO₂) | | |

Advantageous TiO₂ pigments are available, for example, under the trade name Uvinul® TiO₂, and advantageous TiO₂/Fe₂O₃ mixed oxides are available under the trade name Uvinul® TiO₂ A from BASF.

The total amount of inorganic pigments, in particular hydrophobic inorganic micropigments, optionally used in the finished cosmetic or dermatological preparations is advantageously chosen from the range from 0.1 to 30% by weight, preferably 0.1 to 10.0, in particular 0.5 to 6.0% by weight, based on the total weight of the preparations.

In addition to the constituents according to the invention already described, the cosmetic and dermatological preparations according to the invention can comprise further cosmetic active ingredients, auxiliaries and/or additives, as are customarily used in such preparations, e.g. antioxidants, preservatives, bacteriocides, perfumes, antifoams, dyes, pigments which have a coloring action, emollients, moisturizers and/or humectants, fats, oils, waxes or other customary constituents of a cosmetic or dermatological formulation, such as foam stabilizers, electrolytes, organic solvents or silicone derivatives.

Preservatives permitted in food technology, which are listed below with their E number, are to be used advantageously according to the invention.

| | |
|---|---|
| E 200 | Sorbic acid |
| E 201 | Sodium sorbate |
| E 202 | Potassium sorbate |
| E 203 | Calcium sorbate |
| E 210 | Benzoic acid |
| E 211 | Sodium benzoate |
| E 212 | Potassium benzoate |
| E 213 | Calcium benzoate |
| E 214 | Ethyl p-hydroxybenzoate |
| E 215 | p-hydroxybenzoic ethyl |
| | ester Na salt |
| E 216 | n-propyl p-hydroxybenzoate |
| E 217 | p-hydroxybenzoic-n-propyl |
| | ester Na salt |
| E 218 | methyl p-hydroxybenzoate |
| E 219 | p-hydroxybenzoic methyl |
| | ester Na salt |
| E 220 | Sulfur dioxide |
| E 221 | Sodium sulfite |
| E 222 | Sodium hydrogensulfite |
| E 223 | Sodium disulfite |
| E 224 | Potassium disulfite |
| E 226 | Calcium sulfite |
| E 227 | Calcium hydrogen sulfite |
| E 228 | Potassium hydrogen |
| | sulfite |
| E 230 | Biphenyl (Diphenyl) |
| E 231 | Orthophenylphenol |
| E 232 | Sodium |
| | orthophenylphenoxide |
| E 233 | Thiabendazole |
| E 235 | Natamycin |
| E 236 | Formic acid |
| E 237 | Sodium formate |
| E 238 | Calcium formate |
| E 239 | Hexamethylenetetramine |
| E 249 | Potassium nitrite |
| E 250 | Sodium nitrite |
| E 251 | Sodium nitrate |
| E 252 | Potassium nitrate |
| E 280 | Propionic acid |
| E 281 | Sodium propionate |
| E 282 | Calcium propionate |
| E 283 | Potassium propionate |
| E 290 | Carbon dioxide |

Also suitable are preservatives or preservative auxiliaries customary in cosmetics: dibromodicyanobutane (2-bromo-2-bromomethylglutarodinitrile), 3-iodo-2-propinylbutylcarbamate, 2-bromo-2-nitropropane-1,3-diol, imidazolidinylurea, 5-chloro-2-methyl-4-isothiazolin-3-one, 2-chloroacetamide, benzalkonium chloride, benzyl alcohol. +Formaldehyde donors.

Also suitable as preservatives are phenyl hydroxyalkyl ethers, in particular the compounds known under the name phenoxyethanol, because of their bactericidal and fungicidal effects on a number of microorganisms.

Other antimicrobial agents are likewise suitable for being incorporated into the preparations according to the invention. Advantageous substances are, for example,

2,4,4′-trichloro-2′-hydroxydiphenyl ether (Irgasan),

1,6-di(4-chlorophenylbiguanido)hexane (Chlorhexidin),

3,4,4′-trichlorocarbanilide, quaternary ammonium compounds, oil of cloves, mint oil, thyme oil, triethyl citrate, farnesol (3,7,11-trimethyl-2,6,10-dodecatrien-1-ol) and the active ingredients or active ingredient combinations described in the patent laid-open specifications DE-37 40 186, DE-39 38 140, DE-42 04 321, DE-42 29 707, DE-43 09 372, DE-44 11 664, DE-195 41 967, DE-195 43 695, DE-195 43 696, DE-195 47 160, DE-196 02 108, DE-196 02 110, DE-196 02 111, DE-196 31 003, DE-196 31 004 and DE-196 34 019 and the patent specifications DE-42 29 737, DE-42 37 081, DE-43 24 219, DE-44 29 467, DE-44 23 410 and DE-195 16 705. Sodium hydrogencarbonate can also be used advantageously.

For the purposes of the present invention, it is, moreover, advantageous to add other anti-irritative or anti-inflammatory active ingredients to the preparations, in particular batyl alcohol (α-octadecyl glyceryl ether), selachyl alcohol (α-9-octadecenyl glyceryl ether), chimyl alcohol (α-hexadecyl glyceryl ether), bisabolol and/or panthenol.

It is likewise advantageous to add conventional antioxidants to the preparations for the purposes of the present invention. According to the invention, favorable antioxidants can be any antioxidants which are suitable or customary for cosmetic and/or dermatological applications.

The antioxidants are advantageously selected from the group consisting of amino acids (for example glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotenoids, carotenes (e.g. α-carotene, β-carotene, ψ-lycopene) and derivatives thereof, chlorogenic acid and derivatives thereof, aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximines, homocysteine sulfoximine, buthionine sulfones, penta-, hexa-, heptathionine sulfoximine) in very small tolerated doses (e.g. pmol to μmol/kg), also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, furfurylidenesorbitol and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), and coniferyl benzoate of benzoin resin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO4), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) of said active ingredients which are suitable according to the invention.

The amount of antioxidants (one or more compounds) in the preparations is preferably from 0.001 to 30% by weight, particularly preferably 0.05-20% by weight, in particular 1-10% by weight, based on the total weight of the preparation.

If vitamin E and/or derivatives thereof are used as the antioxidant(s), it is advantageous to choose their respective concentrations from the range 0.001-10% by weight, based on the total weight of the formulation.

Preparations according to the present invention can also be used as a basis for cosmetic or dermatological deodorants or antiperspirants. All active ingredients which are common for deodorants or antiperspirants can be used advantageously, for example odor maskers such as the customary perfume constituents, odor absorbers, for example the phyllosilicates described in patent laid-open specification DE-P 40 09 347, and of these, in particular montmorillonite, kaolinite, illite, beidellite, nontronite, saponite, hectorite, bentonite, smectite, and also, for example, zinc salts of ricinoleic acid.

The amount of such active ingredients (one or more compounds) in the preparations according to the invention is preferably 0.001 to 30% by weight, particularly preferably 0.05 to 20% by weight, in particular 1 to 10% by weight, based on the total weight of the preparation.

Further additives may, inter alia, be quaternary surfactants. These compounds contain at least one N atom which is covalently bonded to 4 alkyl or aryl groups. This leads to a positive charge irrespective of the pH. Advantageous compounds are alkylbetaine, alkylamidopropylbetaine and alkylamidopropylhydroxysulfaine. The cationic surfactants used according to the invention can also preferably be chosen from the group of quaternary ammonium compounds, in particular benzyltrialkylammonium chlorides or bromides such as, for example, benzyldimethylstearylammonium chloride, and also alkyltrialkylammonium salts, for example cetyltrimethylammonium chloride or bromide, alkyldimethylhydroxyethylammonium chlorides or bromides, dialkyldimethylammonium chlorides or bromides, alkylamidoethyltrimethylammonium ether sulfates, alkylpyridinium salts, for example lauryl- or cetylpyridinium chloride, imidazoline derivatives and compounds with cationic character, such as amine oxides, for example alkyldimethylamine oxides or alkylaminoethyldimethylamine oxides. In particular, cetyltrimethylammonium salts are to be used advantageously.

In addition, “metal soaps” (i.e. salts of higher fatty acids with the exception of alkali metal salts) are also advantageous oil thickeners for the purposes of the present invention, such as, for example, aluminum stearate, zinc stearate and/or magnesium stearate.

It is likewise advantageous to add amphoteric or zwitterionic surfactants (e.g. cocamidopropylbetain) and moisturizers (e.g. betain) to preparations according to the invention. Examples of amphoteric surfactants to be used advantageously are acyl/dialkylethylenediamine, for example sodium acylamphoacetate, disodium acylamphodipropionate, disodium alkylamphodiacetate, sodium acylamphohydroxypropylsulfonate, disodium acylamphodiacetate and sodium acylamphopropionate, N-alkylamino acids, for example aminopropylalkylglutamide, alkylaminopropionic acid, sodium alkylimidodipropionate and lauroamphocarboxyglycinate.

A surprising property of the preparations according to the invention is that they are very good vehicles for cosmetic or dermatological active ingredients into the skin, preferred active ingredients being the aforementioned antioxidants, which can protect the skin against oxidative stress.

According to the invention, the active ingredients (one or more compounds) can also very advantageously be chosen from the group of lipophilic active ingredients, in particular from the following group:

Acetylsalicylic acid, atropine, azulene, hydrocortisone and derivatives thereof, e.g. hydrocortisone-17-valerate, vitamins of the B and D series, very favorably vitamin B₁, vitamin B₁₂, vitamin D₁, but also bisabolol, unsaturated fatty acids, namely the essential fatty acids (often also called vitamin F), in particular γ-linolenic acid, oleic acid, eicosapentenoic acid, docosahexenoic acid and derivatives thereof, chloramphenicol, caffeine, prostaglandins, thymol, camphor, extracts or other products of a vegetable and animal origin, e.g. evening primrose oil, borrage oil or currant seed oil, fish oils, cod-liver oil and also ceramides and ceramide-like compounds, etc.

It is also advantageous to choose the active ingredients from the group of refatting substances, for example purcellin oil, Eucerit® and Neocerit®.

The active ingredient(s) is/are particularly advantageously chosen from the group of NO synthesase inhibitors, particularly if the preparations according to the invention are to be used for the treatment and prophylaxis of the symptoms of intrinsic and/or extrinsic skin aging and for the treatment and prophylaxis of the harmful effects of ultraviolet radiation on the skin.

A preferred NO synthase inhibitor is nitroarginine.

The active ingredient(s) is/are further advantageously chosen from the group which includes catechins and bile esters of catechins and aqueous or organic extracts from plants or sections of plants which have a content of catechins or bile esters of catechins, such as, for example, the leaves of the Theaceae plant family, in particular of the species Camellia sinensis (green tea). Their typical ingredients (such as, for example, polyphenols or catechins, caffeine, vitamins, sugars, minerals, aminoacids, lipids) are particularly advantageous.

Catechins are a group of compounds which are to be regarded as hydrogenated flavones or anthocyanidines and are derivatives of “catechin” (catechol, 3,3′,4′,5,7-flavanpentol, 2-(3,4-dihydroxyphenyl)chroman-3,5,7-triol). Epicatechin ((2R,3R)-3,3′,4′,5,7-flavanpentol) is also an advantageous active ingredient for the purposes of the present invention.

Preferred active ingredients are also polyphenols and catechins from the group (−)-catechin, (+)-catechin, (−)-catechin gallate, (−)-gallocatechin gallate, (+)-epicatechin, (−)-epicatechin, (−)-epicatechin gallate, (−)-epigallocatechin, (−)-epigallocatechin gallate.

Flavone and its derivatives (also often collectively called “flavones”) are also advantageous active ingredients for the purposes of the present invention. They are characterized by the following basic structure (substitution positions are shown):

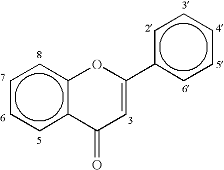

Some of the more important flavones which can also preferably be used in preparations according to the invention are given in table 2 below:

| | OH substitution positions | | | | | | | |
|---|---|---|---|---|---|---|---|---|
| | 3 | 5 | 7 | 8 | 2′ | 3′ | 4′ | 5′ |
| Flavone | − | − | − | − | − | − | − | − |
| Flavonol | + | − | − | − | − | − | − | − |
| Chrysin | − | + | + | − | − | − | − | − |
| Galangin | + | + | + | − | − | − | − | − |
| Apigenin | − | + | + | − | − | − | + | − |
| Fisetin | + | − | + | − | − | + | + | − |
| Luteolin | − | + | + | − | − | + | + | − |
| Kaempferol | + | + | + | − | − | − | + | − |
| Quercetin | + | + | + | − | − | + | + | − |
| Morin | + | + | + | − | + | − | + | − |
| Robinetin | + | − | + | − | − | + | + | + |
| Gossypetin | + | + | + | + | − | + | + | − |
| Myricetin | + | + | + | − | − | + | + | + |

In nature, flavones are usually in glycosylated form.

According to the invention, the flavonoids are preferably chosen from the group of substances of the generic structural formula

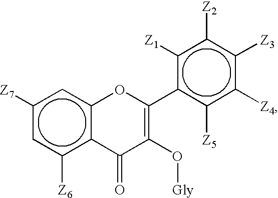

where Z₁ to Z₇, independently of one another, are chosen from the group consisting of H, OH, alkoxy and hydroxyalkoxy, where the alkoxy and hydroxyalkoxy groups can be branched or unbranched and have 1 to 18 carbon atoms, and where Gly is chosen from the group of mono- and oligoglycoside radicals.

According to the invention, the flavonoids can, however, also advantageously be chosen from the group of substances of the generic structural formula

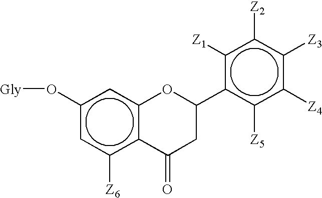

where Z₁ to Z₆, independently of one another, are chosen from the group consisting of H, OH, alkoxy and hydroxyalkoxy, where the alkoxy and hydroxyalkoxy groups may be branched or unbranched and have 1 to 18 carbon atoms, where Gly is chosen from the group mono and oligoglycoside radicals.

Preferably, such structures can be chosen from the group of substances of the generic structural formula

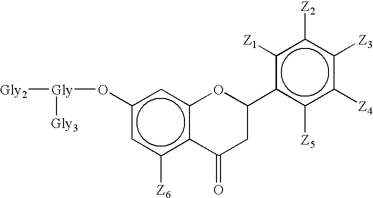

where Gly₁, Gly₂ and Gly₃, independently of one another, are monoglycoside radicals. Gly₂ and Gly₃ may also, individually or together, represent saturations by hydrogen atoms.

Preferably, Gly₁, Gly₂ and Gly₃, independently of one another, are chosen from the group of hexosyl radicals, in particular the rhamnosyl radicals and glucosyl radicals. However, hexosyl radicals, for example allosyl, altrosyl, galactosyl, gulosyl, idosyl, mannosyl and talosyl, can also be used advantageously in some circumstances. It may also be advantageous according to the invention to use pentosyl radicals.

Z₁ to Z₅ are, independently of one another, advantageously chosen from the group consisting of H, OH, methoxy, ethoxy and 2-hydroxyethoxy, and the flavone glycosides have the structure

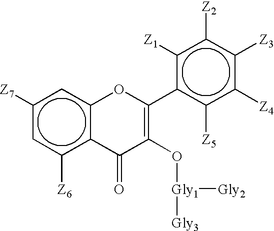

The flavone glycosides according to the invention are particularly advantageously chosen from the group given by the following structure:

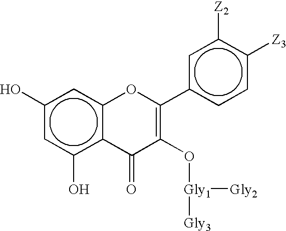

where Gly₁, Gly₂ and Gly₃, independently of one another, are monoglycoside radicals. Gly₂ and Gly₃ can also, individually or together, represent saturations by hydrogen atoms.

Preferably, Gly₁, Gly₂ and Gly₃, independently of one another, are chosen from the group of hexosyl radicals, in particular of rhamnosyl radicals and glucosyl radicals. However, other hexosyl radicals, for example allosyl, altrosyl, galactosyl, gulosyl, idosyl, mannosyl and talosyl, can also advantageously be used in some circumstances. It may also be advantageous according to the invention to use pentosyl radicals.

For the purposes of the present invention, it is particularly advantageous to choose the flavone glucoside(s) from the group consisting of α-glucosylrutin, α-glucosylmyricetin, α-glucosylisoquercitrin, α-glucosylisoquercetin and α-glucosylquercitrin.

Particular preference is given according to the invention to α-glucosylrutin.

Also advantageous according to the invention are naringin (aurantin, naringenin-7-rhamno-glucoside), hesperidin 3′,5,7-trihydroxy-4′-methoxyflavanone-7-rutinoside, hesperidoside, hesperetin-7-O-rutinoside), rutin (3,3′,4′,5,7-pentahydroxyflavone-3-rutinoside, quercetin-3-rutinoside, sophorin, birutan, rutabion, taurutin, phytomelin, melin), troxerutin (3,5-dihydroxy-3′,4′,7-tris(2-hydroxyethoxy)flavone-3-(6-O-(6-deoxy-α-L-mannopyranosyl)-β-D-glucopyranoside)), monoxerutin (3,3′,4′,5-tetrahydroxy-7-(2-hydroxyethoxy)flavone-3-(6-O-(6-deoxy-α-L-mannopyranosyl)-β-D-glucopyranoside)), dihydrorobinetin (3,3′,4′,5′,7-pentahydroxyflavanone), taxifolin (3,3′,4′,5,7-pentahydroxyflavanone), eriodictyol-7-glucoside (3′,4′,5,7-tetrahydroxyflavanone-7 glucoside), flavanomarein (3′,4′,7,8-tetrahydroxyflavanone-7 glucoside) and isoquercetin (3,3′,4′,5,7-pentahydroxyflavanone-3-(β-D-glucopyranoside).

It is also advantageous to choose the active ingredient(s) from the group of ubiquinones and plastoquinones.

Ubiquinones are characterized by the structural formula

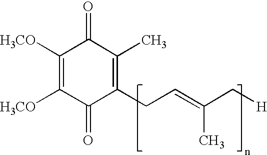

and are the most widespread and thus the most investigated bioquinones. Ubiquinones are referred to, depending on the number of isoprene units linked in the side chain, as Q-1, Q-2, Q-3 etc., or according to the number of carbon atoms, as U-5, U-10, U-15etc. They preferably arise with certain chain lengths, e.g. in some microorganisms and yeasts where n=6. In most mammals including man, Q10 predominates.

Coenzyme Q10 is particularly advantageous and is characterized by the following structural formula:

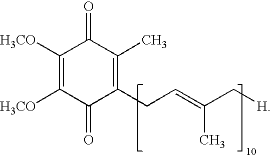

Plastoquinones have the general structural formula

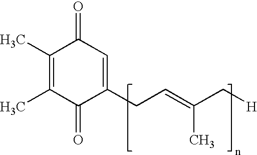

Plastoquinones differ in the number n of isoprene radicals and are referred to accordingly, e.g. PQ-9 (n=9). In addition, other plastoquinones with varying substituents on the quinone ring exist.

Creatine and/or creatine derivatives are preferred active ingredients for the purposes of the present invention. Creatine is characterized by the following structure:

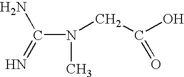

Preferred derivatives are creatine phosphate and creatine sulfate, creatine acetate, creatine ascorbate and the derivatives esterified at the carboxyl group with mono- or polyfunctional alcohols.

A further advantageous active ingredient is L-carnitine [3-hydroxy-4-(trimethylammonio)butyrobetaine]. Acylcarnitines, chosen from the group of substances of the following general structural formula

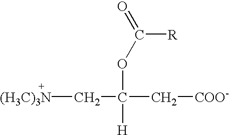

where R is chosen from the group of branched and unbranched alkyl radicals having up to 10 carbon atoms, are advantageous active ingredients for the purposes of the present invention. Preference is given to propionylcarnitine and, in particular, acetylcarnitine. Both enantiomers (D and L form) are to be used advantageously for the purposes of the present invention. It may also be advantageous to use any enantiomer mixtures, for example a racemate of D and L form.

Further advantageous active ingredients are sericoside, pyridoxol, vitamin K and biotin and aroma substances.

The list of said active ingredients and active ingredient combinations which can be used in the preparations according to the invention is, of course, not intended to be limiting. The active ingredients can be used individually or in any combinations with one another.

The amount of such active ingredients (one or more compounds) in the preparations according to the invention is preferably 0.001 to 30% by weight, particularly preferably 0.05 to 20% by weight, in particular 0.1 to 10% by weight, based on the total weight of the preparation.

In some instances, it may also be advantageous for the purposes of the present invention to incorporate dyes and/or color pigments into the preparations according to the invention.

The dyes and color pigments can be chosen from the corresponding positive list of the Cosmetics Directive or the EC list of cosmetic colorants. In most cases they are identical to the dyes approved for foods. Advantageous color pigments are, for example, titanium dioxide, mica, iron oxides (e.g. Fe₂O₃, Fe₃O₄, FeO(OH)) and/or tin oxide. Advantageous dyes are, for example, carmine, Berlin blue, chrome oxide green, ultramarine blue and/or manganese violet. It is particularly advantageous to choose the dyes and/or color pigments from the following list. The Colour Index Numbers (CIN) are taken from the Rowe Colour Index, 3rd Edition, Society of Dyers and Colourists, Bradford, England, 1971.

| Chemical or other name | CIN | Color |
|---|---|---|
| Pigment Green | 10006 | green |
| Acid Green 1 | 10020 | green |
| 2,4-Dinitrohydroxynaphthalene-7-sulfonic acid | 10316 | yellow |
| Pigment Yellow 1 | 11680 | yellow |
| Pigment Yellow 3 | 11710 | yellow |
| Pigment Orange 1 | 11725 | orange |
| 2,4-Dihydroxyazobenzene | 11920 | orange |
| Solvent Red 3 | 12010 | red |
| 1-(2′-Chloro-4′-nitro-1′-phenylazo)-2-hydroxy- | 12085 | red |
| naphthalene | | |
| Pigment Red 3 | 12120 | red |
| Ceres red; Sudan red; Fat Red G | 12150 | red |
| Pigment Red 112 | 12370 | red |
| Pigment Red 7 | 12420 | red |
| Pigment Brown 1 | 12480 | brown |
| 4-(2′-Methoxy-5′-sulfodiethylamido-1′-phenylazo)- | 12490 | red |
| 3-hydroxy-5″-chloro-2″,4″-dimethoxy-2-naphthanilide | | |
| Disperse Yellow 16 | 12700 | yellow |
| 1-(4-Sulfo-1-phenylazo)-4-aminobenzene-5-sulfonic | 13015 | yellow |
| acid | | |
| 2,4-Dihydroxyazobenzene-4′-sulfonic acid | 14270 | orange |
| 2-(2,4-Dimethylphenylazo-5-sulfo)-1-hydroxy- | 14700 | red |
| naphthalene-4-sulfonic acid | | |
| 2-(4-Sulfo-1-naphthylazo)-1-naphthol-4-sulfonic | 14720 | red |
| acid | | |
| 2-(6-Sulfo-2,4-xylylazo)-1-naphthol-5-sulfonic | 14815 | red |
| acid | | |
| 1-(4′-Sulfophenylazo)-2-hydroxynaphthalene | 15510 | orange |
| 1-(2-Sulfo-4-chloro-5-carboxy-1-phenylazo)- | 15525 | red |
| 2-hydroxynaphthalene | | |
| 1-(3-Methylphenylazo-4-sulfo)-2-hydroxynaphthalene | 15580 | red |
| 1-(4′,(8′)-Sulfonaphthylazo)-2-hydroxynaphthalene | 15620 | red |
| 2-Hydroxy-1,2′-azonaphthalene-1′-sulfonic acid | 15630 | red |
| 3-Hydroxy-4-phenylazo-2-naphthylcarboxylic | 15800 | red |
| acid | | |
| 1-(2-Sulfo-4-methyl-1-phenylazo)-2-naphthyl- | 15850 | red |
| carboxylic acid | | |
| 1-(2-Sulfo-4-methyl-5-chloro-1-phenylazo)-2- | 15865 | red |
| hydroxynaphthalene-3-carboxylic acid | | |
| 1-(2-Sulfo-1-naphthylazo)-2-hydroxynaphthalene- | 15880 | red |
| 3-carboxylic acid | | |
| 1-(3-Sulfo-1-phenylazo)-2-naphthol-6-sulfonic | 15980 | orange |
| acid | | |
| 1-(4-Su1fo-1-phenylazo)-2-naphthol-6-sulfonic | 15985 | yellow |
| acid | | |
| Allura Red | 16035 | red |
| 1-(4-Sulfo-1-naphthylazo)-2-naphthol-3,6-disulfonic | 16185 | red |
| acid | | |
| Acid Orange 10 | 16230 | orange |
| 1-(4-Sulfo-1-naphthylazo)-2-naphthol-6,8-disulfonic | 16255 | red |
| acid | | |
| 1-(4-Sulfo-1-naphthylazo)-2-naphthol-3,6,8-trisulfonic | 16290 | red |
| acid | | |
| 8-Amino-2-phenylazo-1-naphthol-3,6-disulfonic | 17200 | red |
| acid | | |
| Acid Red 1 | 18050 | red |
| Acid Red 155 | 18130 | red |
| Acid Yellow 121 | 18690 | yellow |
| Acid Red 180 | 18736 | red |
| Acid Yellow 11 | 18820 | yellow |
| Acid Yellow 17 | 18965 | yellow |
| 4-(4-Sulfo-1-phenylazo)-1-(4-sulfophenyl)-5-hydroxy- | 19140 | yellow |
| pyrazolone-3-carboxylic acid | | |
| Pigment Yellow 16 | 20040 | yellow |
| 2,6-(4′-Sulfo-2″,4″-dimethyl)bisphenylazo)- | 20170 | orange |
| 1,3-dihydroxybenzene | | |
| Acid Black 1 | 20470 | black |
| Pigment Yellow 13 | 21100 | yellow |
| Pigment Yellow 83 | 21108 | yellow |
| Solvent Yellow | 21230 | yellow |
| Acid Red 163 | 24790 | red |
| Acid Red 73 | 27290 | red |
| 2-[4′-(4″-Sulfo-1″-phenylazo)-7′-sulfo-1′-naphthylazo]- | 27755 | black |
| 1-hydroxy-7-aminonaphthalene-3,6-disulfonic | | |
| acid | | |
| 4′-[(4″-Sulfo-1″-phenylazo)-7′-sulfo-1′naphthylazo]- | 28440 | black |
| 1-hydroxy-8-acetylaminonaphthalene- | | |
| 3,5-disulfonic acid | | |
| Direct Orange 34, 39, 44, 46, 60 | 40215 | orange |
| Food Yellow | 40800 | orange |
| trans-β-Apo-8′-carotinaldehyde (C₃₀) | 40820 | orange |
| trans-Apo-8′-carotinic acid (C₃₀)-ethyl ester | 40825 | orange |
| Canthaxanthin | 40850 | orange |
| Acid Blue 1 | 42045 | blue |
| 2,4-Disulfo-5-hydroxy-4′-4″-bis(diethylamino) | 42051 | blue |
| triphenylcarbinol | | |
| 4-[(4-N-Ethyl-p-sulfobenzylamino)phenyl(4-hydroxy- | 42053 | green |
| 2-sulfophenyl)(methylene)-1-(N-ethyl- | | |
| N-p-sulfobenzyl)-2,5-cyclohexadienimine] | | |
| Acid Blue 7 | 42080 | blue |
| (N-Ethyl-p-sulfobenzylamino)phenyl(2-sulfophenyl) | 42090 | blue |
| methylene-(N-ethyl-N-p-sulfobenzyl)Δ^{2,5}- | | |
| cyclohexadienimine | | |
| Acid Green 9 | 42100 | green |
| Diethyldisulfobenzyldi-4-amino-2-chloro- | 42170 | green |
| di-2-methyl-fuchsonimmonium | | |
| Basic Violet 14 | 42510 | violet |
| Basic Violet 2 | 42520 | violet |
| 2′-Methyl-4′-(N-ethyl-N-m-Sulfobenzyl)amino- | 42735 | blue |
| 4″-(N-diethyl)amino-2-methyl-N-ethyl- | | |
| N-m-sulfobenzylfuchsonimmonium | | |
| 4′-(N-Dimethyl) amino-4″-(N-phenyl)aminonaphtho- | 44045 | blue |
| N-dimethyl-fuchsonimmonium | | |
| 2-Hydroxy-3,6-disulfo-4,4′-bisdimethylamino- | 44090 | green |
| naphtho-fuchsonimmonium | | |
| Acid Red 52 | 45100 | red |
| 3-(2′-Methylphenylamino)-6-(2′-methyl-4′- | 45190 | violet |
| sulfophenylamino)-9-(2″-carboxyphenyl)xanthenium | | |
| salt | | |
| Acid Red 50 | 45220 | red |
| Phenyl-2-oxyfluorone-2-carboxylic acid | 45350 | yellow |
| 4,5-Dibromofluorescein | 45370 | orange |
| 2,4,5,7-Tetrabromofluorescein | 45380 | red |
| Solvent Dye | 45396 | orange |
| Acid Red 98 | 45405 | red |
| 3′,4′,5′,6′-Tetrachloro-2,4,5,7-tetrabromofluorescein | 45410 | red |
| 4,5-Diiodofluorescein | 45425 | red |
| 2,4,5,7-Tetraiodofluorescein | 45430 | red |
| Quinophthalone | 47000 | yellow |
| Quinophthalonedisulfonic acid | 47005 | yellow |
| Acid Violet 50 | 50325 | violet |
| Acid Black 2 | 50420 | black |
| Pigment Violet 23 | 51319 | violet |
| 1,2-Dioxyanthraquinone, calcium-aluminum | 58000 | red |
| complex | | |
| 3-Oxypyrene-5,8,10-sulfonic acid | 59040 | green |
| 1-Hydroxy-4-N-phenylaminoanthraquinone | 60724 | violet |
| 1-Hydroxy-4-(4′-methylphenylamino)anthraquinone | 60725 | violet |
| Acid Violet 23 | 60730 | violet |
| 1,4-Di(4′-methylphenylamino)anthraquinone | 61565 | green |
| 1,4-Bis(o-sulfo-p-toluidino)anthraquinone | 61570 | green |
| Acid Blue 80 | 61585 | blue |
| Acid Blue 62 | 62045 | blue |
| N,N′-Dihydro-1,2,1′,2′-anthraquinone azine | 69800 | blue |
| Vat Blue 6; Pigment Blue 64 | 69825 | blue |
| Vat Orange 7 | 71105 | orange |
| Indigo | 73000 | blue |
| Indigo-disulfonic acid | 73015 | blue |
| 4,4′-Dimethyl-6,6′-dichlorothioindigo | 73360 | red |
| 5,5′-Dichloro-7,7′-dimethylthioindigo | 73385 | violet |
| Quinacridone Violet 19 | 73900 | violet |
| Pigment Red 122 | 73915 | red |
| Pigment Blue 16 | 74100 | blue |
| Phthalocyanine | 74160 | blue |
| Direct Blue 86 | 74180 | blue |
| Chlorinated Phthalocyanines | 74260 | green |
| Natural Yellow 6,19; Natural Red 1 | 75100 | yellow |
| Bixin, Nor-Bixin | 75120 | orange |
| Lycopene | 75125 | yellow |
| trans-alpha-, beta- and gamma-carotene | 75130 | orange |
| Keto- and/or hydroxyl derivates of carotene | 75135 | yellow |
| Guanine or pearlizing agent | 75170 | white |
| 1,7-Bis(4-hydroxy-3-methoxyphenyl)-1,6-heptadiene- | 75300 | yellow |
| 3,5-dione | | |
| Complex salt (Na, Al, Ca) of carminic acid | 75470 | red |
| Chlorophyll a and b; copper compounds of | 75810 | green |
| chlorophylls and Chlorophyllins | | |
| Aluminum | 77000 | white |
| Hydrated alumina | 77002 | white |
| Hydrous aluminum silicates | 77004 | white |
| Ultramarine | 77007 | blue |
| Pigment Red 101 und 102 | 77015 | red |
| Barium sulfate | 77120 | white |
| Bismuth oxychloride and its mixtures with mica | 77163 | white |
| Calcium carbonate | 77220 | white |
| Calcium sulfate | 77231 | white |
| Carbon | 77266 | black |
| Pigment Black 9 | 77267 | black |
| Carbo medicinalis vegetabilis | 77268 | black |
| Chromium oxide | 77288 | green |
| Chromium oxide, hydrous | 77289 | green |
| Pigment Blue 28, Pigment Green 14 | 77346 | green |
| Pigment Metal 2 | 77400 | brown |
| Gold | 77480 | brown |
| Iron oxides and hydroxides | 77489 | orange |
| Iron oxide | 77491 | red |
| Iron oxide, hydrated | 77492 | yellow |
| Iron oxide | 77499 | black |
| Mixtures of iron (II) and iron(III)hexacyanoferrate | 77510 | blue |
| Pigment White 18 | 77713 | white |
| Manganese ammonium diphosphate | 77742 | violet |
| Manganese phosphate; Mn₃(PO₄)₂.7 H₂O | 77745 | red |
| Silver | 77820 | white |
| Titanium dioxide and its mixtures with mica | 77891 | white |
| Zinc oxide | 77947 | white |
| 6,7-Dimethyl-9-(1′-D-ribityl)-isoalloxazine, | | yellow |
| lactoflavine | | |
| Sugar coloring | | brown |
| Capsanthin, capsorubin | | orange |
| Betanin | | red |
| Benzopyrylium salts, Anthocyans | | red |
| Aluminum, zinc, magnesium and calcium stearate | | white |
| Bromothymol blue | | blue |
| Bromocresol green | | green |
| Acid Red 195 | | red |

Also advantageous are oil-soluble natural dyes, such as, for example, paprika extracts, β-carotene or cochenille. Also advantageous for the purposes of the present invention are gel creams with a content of pearlescent pigments. Preference is given in particular to the types of pearlescent pigments listed below:

Natural pearlescent pigments, such as, for example

“pearl essence” (guanine/hypoxanthin mixed crystals from fish scales), “mother of pearl” (ground mussel shells) and monocrystalline pearlescent pigments, such as, for example, bismuth oxychloride (BiOCl)

layer substrate pigments: e.g. mica/metal oxide.

Bases for pearlescent pigments are, for example, pulverulent pigments or castor oil dispersions of bismuth oxychloride and/or titanium dioxide, and bismuth oxychloride and/or titanium dioxide on mica. The luster pigment listed under CIN 77163, for example, is particularly advantageous.

Also advantageous are, for example, the following types of pearlescent pigment based on mica/metal oxide:

| | Coating/layer | |
|---|---|---|
| Group | thickness | Color |
| Silver-white pearlescent | TiO₂: 40-60 nm | silver |
| pigments | | |
| Interference pigments | TiO₂: 60-80 nm | yellow |
| | TiO₂: 80-100 nm | red |
| | TiO₂: 100-140 nm | blue |
| | TiO₂: 120-160 nm | green |
| Color luster pigments | Fe₂O₃ | bronze |
| | Fe₂O₃ | copper |
| | Fe₂O₃ | red |
| | Fe₂O₃ | red-violet |
| | Fe₂O₃ | red-green |
| | Fe₂O₃ | black |
| Combination pigments | TiO₂/Fe₂O₃ | gold |
| | | shades |
| | TiO₂/Cr₂O₃ | green |
| | TiO₂/Berlin blue | deep blue |
| | TiO₂/carmine | red |

Particular preference is given, for example, to the pearlescent pigments available from Merck under the trade names Timiron, Colorona or Dichrona.

The list of given pearlescent pigments is not of course intended to be limiting. Pearlescent pigments which are advantageous for the purposes of the present invention are obtainable in numerous ways known per se. For example, other substrates apart from mica can be coated with further metal oxides, such as, for example, silica and the like. SiO₂ particles coated with, for example, TiO₂ and Fe₂O₃ (“ronaspheres”), which are sold by Merck and are particularly suitable for the optical reduction of fine lines are advantageous.

It can moreover be advantageous to dispense completely with a substrate such as mica. Particular preference is given to pearlescent pigments prepared using SiO₂. Such pigments, which may also additionally have goniochromatic effects, are available, for example, under the trade name Sicopearl Fantastico from BASF.

Pigments from Engelhard/Mearl based on calcium sodium borosilicate which have been coated with titanium dioxide can also be used advantageously. These are available under the name Reflecks. In addition to the color, as a result of their particle size of from 40 μm to 180 μm, they have a glitter effect. In addition, also particularly advantageous are effect pigments which are available under the trade name Metasomes Standard/Glitter in various colors (yellow, red, green, blue) from Flora Tech. The glitter particles are present here in the mixtures with various auxiliaries and dyes (such as, for example, the dyes with the Colour Index (CI) Numbers 19140, 77007, 77289, 77491).

The dyes and pigments may be present either individually or in a mixture, and can be mutually coated with one another, different coating thicknesses generally giving rise to different color effects. The total amount of dyes and color-imparting pigments is advantageously chosen from the range from e.g. 0.1% by weight to 30% by weight, preferably from 0.5 to 15% by weight, in particular from 1.0 to 10% by weight, in each case based on the total weight of the preparations.

The preparations according to the invention are prepared under conditions familiar to the person skilled in the art. Generally the constituents of the oil phase or of the water phase are mixed together and heated separately, and then they are combined together with stirring and, particularly advantageously, with homogenization, very particularly advantageously with stirring with moderate to high input of energy, advantageously using a gear rim dispersing machine at a rotary number up to at most 10000 rpm, preferably from 2500 to 7700 rpm.

The present invention will be illustrated in more detail by reference to the examples below (formulation recipes).

### EXAMPLE 1 TO 5

| | Ex. 1 | Ex. 2 | Ex. 3 | Ex. 4 | Ex. 5 |
|---|---|---|---|---|---|
| Sodium carbomer | | 0.2 | | | |
| Acrylates/C₁₀-C₃₀ alkyl | 0.3 | 0.2 | 0.6 | | |
| acrylate crosspolymer | | | | | |
| Hydroxypropylcellulose | | | | 1.0 | 1.50 |
| Xanthan gum | | 0.6 | 0.2 | 1.0 | 1.0 |
| Compound I | 3.5 | 3.5 | 1.5 | 6.0 | 5.0 |
| Dioctylbutamidotriazone | 2.0 | 2.0 | 1.0 | | |
| Ethylhexyltriazone | 4.0 | 4.0 | 5.0 | | |
| Anisotriazine | 1.0 | 0.5 | | 2.0 | 2.5 |
| Bisoctyltriazole | | | | 6.0 | |
| Drometrizole trisiloxane | | | | | |
| Phenylbenzimidiazole- | 2.0 | | | 1.0 | |
| sulfonic acid | | | | | |
| Bisimidazylate | | | | 1.0 | |
| Terephthalylidene- | | | | 0.2 | |
| dicamphorsulfonic acid | | | | | |
| Ethylhexyl | 7.5 | 10.0 | | 5.0 | |
| methoxycinnamate | | | | | |
| Octocrylene | | | | | 5.0 |
| Dimethicone | | | | 4.0 | |
| diethylbenzalmalonate | | | | | |
| Ethylhexyl salicylate | | | | | |
| Homosalate | | | | | |
| Butylmethoxydibenzoyl- | 1.0 | 1.0 | 4.0 | | |
| methane | | | | | |
| Titanium dioxide | 1.0 | 4.0 | | | |
| Zinc oxide | | | | 4.0 | |
| Caprylic/capric | | | 2.0 | | |
| triglyceride | | | | | |
| Hydrogenated coco- | | | 3.0 | | |
| glycerides | | | | | |
| C12-15 alkyl benzoate | 2.0 | 2.5 | 3.0 | | |
| Dicaprylyl ether | | 4.0 | | | |
| Butylene glycol | 4.0 | | 2.0 | 6.0 | |
| dicaprylate/dicaprate | | | | | |
| Dicaprylyl carbonate | | 2.0 | | | |
| Cetyldimethicone | 2.0 | 0.5 | 1.0 | | |
| Shea butter | | 2.0 | | | |
| PVP hexadecene copolymer | 0.5 | | 0.05 | 0.5 | |
| Glycerol | 3.0 | 7.5 | | 7.5 | 2.5 |
| Tocopherol | | 0.5 | 0.75 | | 0.2 |
| Trisodium EDTA | 1.0 | 0.5 | 0.5 | 1.0 | 1.5 |
| Sodium citrate | | 0.2 | | | |
| Citric acid | | 0.1 | | 0.1 | 0.1 |
| DMDM hydantoin | | 0.6 | | 0.2 | |
| Methylparaben | 0.5 | | 0.3 | 0.15 | |
| Phenoxyethanol | 0.5 | 0.4 | 0.4 | 1.0 | 0.60 |
| Ethanol | 3.0 | 2.0 | 3.0 | | 1.0 |
| Perfume | 0.2 | | | 0.2 | 0.2 |
| Water | ad. | ad. | ad. | ad. | ad. |
| | 100 | 100 | 100 | 100 | 100 |

### EXAMPLES 6 TO 10

| | Ex. 6 | Ex. 7 | Ex. 8 | Ex. 9 | Ex. 10 |
|---|---|---|---|---|---|
| Sodium carbomer | 0.5 | | | 1.5 | |
| Acrylates/C₁₀-C₃₀ alkyl | | 0.4 | 0.1 | | 0.75 |
| aacrylate crosspolymer | | | | | |
| Hydroxypropylcellulose | | | 0.5 | | 0.25 |
| Xanthan gum | 0.2 | 0.4 | | | |
| Compound I | 0.5 | 2.5 | 1.0 | 2.0 | 6.0 |
| Dioctylbutamidotriazone | 1.0 | | 2.0 | | |
| Ethylhexyltriazone | | 2.0 | | 2.0 | |
| Anisotriazine | 1.0 | 0.2 | 3.0 | 1.0 | |
| Bisoctyltriazole | | | | | 8.0 |
| Drometrizole | | | | | 4.0 |
| trisiloxane | | | | | |
| Phenylbenzimidazole- | | 1.5 | | | |
| sulfonic acid | | | | | |
| Bisimidazylate | | | 1.5 | | |
| Terephthalylidene | | | | | 0.5 |
| dicamphorsulfonic acid | | | | | |
| Ethylhexyl | | 7.5 | 5.0 | 10.0 | |
| methoxycinnamate | | | | | |
| Octocrylene | 10.0 | | 5.0 | | 5.0 |
| Dimethicone | | | | | 2.5 |
| diethylbenzalmalonate | | | | | |
| Ethylhexyl salicylate | | | 3.5 | 5.0 | |
| Homosalate | | | 4.0 | | |
| Butylmethoxydibenzoyl- | 0.5 | | | | |
| methane | | | | | |
| Titanium dioxide | 1.5 | 2.0 | 1.0 | | 2.5 |
| Zinc oxide | | | 1.0 | | 0.5 |
| Caprylic/capric | | | | | |
| triglyceride | | | | | |
| Hydrogenated | | | | | |
| cocoglycerides | | | | | |
| C12-15 alkyl benzoate | | | | | 5.0 |
| Dicaprylyl ether | | | | | 7.5 |
| Butylene glycol | | | | | |
| dicaprylate/dicaprate | | | | | |
| Dicaprylyl carbonate | | 7.5 | | | |
| Cetyldimethicone | | | | | |
| Shea butter | | | | | 3.0 |
| PVP hexadecene | 0.5 | | 0.75 | | 1.0 |
| copolymer | | | | | |
| Glycerol | 5.0 | | 10.0 | | |
| Tocopherol | 0.3 | | 1.5 | | 1.0 |
| Trisodium EDTA | 0.5 | | 0.1 | 0.5 | |
| Sodium citrate | | | 0.3 | | |
| Citric acid | | | 0.15 | | |
| DMDM hydantoin | | | | 0.3 | 0.15 |
| Methylparaben | | 0.4 | | | |
| Phenoxyethanol | | 1.0 | | | |
| Ethanol | 7.5 | | 5.0 | | 7.0 |
| Perfume | | 0.25 | | 0.2 | |
| Water | ad. 100 | ad. 100 | ad. 100 | ad. 100 | ad. 100 |

| | Ex. | Ex. | Ex. | Ex. | Ex. | Ex. |
|---|---|---|---|---|---|---|
| | 11 | 12 | 13 | 14 | 15 | 16 |
| Sodium carbomer | 0.5 | 1.5 | 1.0 | | | 0.5 |
| Acrylates/C₁₀-C₃₀ | 1.0 | | | 0.75 | 1.0 | |
| alkyl acrylate | | | | | | |
| crosspolymer | | | | | | |
| Hydroxypropyl- | | | 0.4 | 1.0 | | 1.0 |
| cellulose | | | | | | |
| Xanthan gum | | 0.6 | 0.2 | 1.0 | 1.0 | |
| Compound I | 4.0 | 0.5 | 3.0 | 2.0 | 4.0 | 1.5 |
| Dioctylbutamidotriazone | 2.0 | 2.0 | | 2.0 | | 1.0 |
| Ethylhexyltriazone | | 4.0 | 5.0 | 4.0 | | |
| Anisotriazine | 1.0 | | | 1.0 | 2.5 | 1.0 |
| Bisoctyltriazole | | | 4.0 | | | |
| Drometrizole | | 3.0 | | | | |
| trisiloxane | | | | | | |
| Phenylbenzimidazole | 2.0 | | | 1.0 | | |
| sulfonic acid | | | | | | |
| Bisimidazylate | | | 1.5 | | 3.5 | |
| Terephthalylidene- | | | | 0.2 | | 1.0 |
| dicamphorsulfonic | | | | | | |
| acid | | | | | | |
| Ethylhexyl | | 10.0 | | 5.0 | | |
| methoxycinnamate | | | | | | |
| Octocrylene | 10.0 | | | | 5.0 | |
| Dimethicone | | | | 4.0 | | |
| diethylbenzal- | | | | | | |
| malonate | | | | | | |
| Ethylhexyl | | | | | | 5.0 |
| salicylate | | | | | | |
| Homosalate | | | | 5.0 | | |
| Butylmethoxy- | 1.0 | 1.0 | 4.0 | | | 0.5 |
| dibenzoylmethane | | | | | | |
| Titanium dioxide | 1.0 | 4.0 | | | | 1.5 |
| Zinc oxide | | | | 4.0 | | |
| Caprylic/capric | | | 2.0 | | | |
| triglyceride | | | | | | |
| Paraffin oil | | | | 1.0 | | |
| C₁₂-C₁₅ alkyl | 2.0 | 2.5 | 3.0 | | | |
| benzoate | | | | | | |
| Dicaprylyl ether | | 4.0 | | | | |
| Isohexadecene | 4.0 | | 2.0 | 6.0 | | |
| Dicaprylyl | | 2.0 | | | | |
| carbonate | | | | | | |
| Dibutyl adipate | 2.0 | 0.5 | 1.0 | | | |
| Cylomethicone | | | | 3.0 | | |
| Jojoba oil | | 2.0 | | | | |
| PVP hexadecene | 0.5 | | 0.05 | 0.5 | | 0.5 |
| copolymer | | | | | | |
| Butylene glycol | 3.0 | 7.5 | | 7.5 | 2.5 | 5.0 |
| Ascorbyl palmitate | | 0.5 | 0.75 | | 0.2 | 0.3 |
| Octoxyglycerol | | 1.0 | | 0.5 | | 1.0 |
| Glycine soya | | | 2.0 | | | 1.5 |
| Trisodium EDTA | 1.0 | 0.5 | 0.5 | | 1.5 | 0.5 |
| Sodium hydroxide | 1.0 | 0.2 | 0.25 | | | |
| solution | | | | | | |
| Iodopropyl | | 0.6 | | 0.2 | | |
| butylcarbamate | | | | | | |
| Phenoxyethanol | | 0.4 | | 1.0 | | |
| Ethanol | 5.0 | 2.0 | 7.0 | | | |
| Perfume | 0.2 | | | 0.2 | 0.2 | |
| Water | ad. | ad. | ad. | ad. | ad. | ad. |
| | 100 | 100 | 100 | 100 | 100 | 100 |

We claim:

1. A cosmetic or dermatological preparation which represents a finely disperse system of the oil-in-water type, comprising
a) an oil phase,
b) a water phase,
c) one or more stabilizers,
d) at most 2.00% by weight of one or more emulsifiers,
e) an amino-substituted hydroxybenzophenone compound of the formula I.

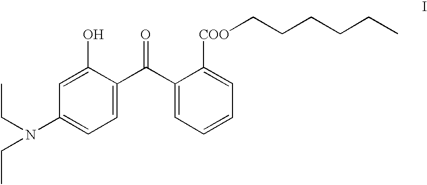

2. A preparation as claimed in claim 1, wherein the content of one or more emulsifiers is between 0.5% by weight and 1.5% by weight, based on the total weight of the preparation.

3. A preparation as claimed in either of claims 1 and 2, which is a hydrodispersion.

4. A preparation as claimed in any of claims 1 to 3, wherein the stabilizer(s) is/are chosen from the group consisting of methylcelluloses, ethylcelluloses, xanthan, carrageen, copolymers of C₁₀-C₃₀-alkyl acrylates and one or more monomers of acrylic acid, polyurethanes and phyllosilicates.

5. A preparation as claimed in claim 4, wherein ethylcelluloses are used as stabilizer.

6. A preparation as claimed in any of claims 1 to 5, wherein the content of one or more stabilizers in the preparation is 0.1 to 10% by weight, based on the total weight of the preparation.
